SEQUENCE LISTING (53 sequences)

SEQ ID NO: 1 - protein, 115 aa, Lama glama
EVKLQQSGGGLVQPGGSLTLSCVTSGFTFSTYGMDWVRQAPGKGFEWVASISFGGGITNYGDFVKGRFTISRDNAKNTLYLHMNGLKPDDTAVYYCQMGSKRGQGTQVTVSSEPQ

SEQ ID NO: 2 - protein, 118 aa, Lama glama
EVKLQESGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 3 - protein, 118 aa, Lama glama
QVKLQQSGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 4 - protein, 120 aa, Lama glama
EVQLQQSGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTQVTVSSEPQ

SEQ ID NO: 5 - protein, 120 aa, Lama glama
EVKLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTQVTVSSEPQ

SEQ ID NO: 6 - protein, 120 aa, Lama glama
EVKLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRANWGQGTQVTVSSEPQ

SEQ ID NO: 7 - protein, 126 aa, Lama glama
EVKLQESGGGLVQPGKSLRLSCEASARAAGVGWFRQAPGLEREYVGSFLWNTGRTHYPESLKDRFTISKDNAKNTVYLQINSLKPEDTAIYYCAAVQLPIRTSLTEPATYTFWGQGTQVTVASEPQ

SEQ ID NO: 8 - protein, 118 aa, Lama glama; identical to SEQ ID NO: 2

SEQ ID NO: 9 - protein, 120 aa, Lama glama; identical to SEQ ID NO: 4

SEQ ID NO: 10 - protein, 120 aa, Lama glama; identical to SEQ ID NO: 6

SEQ ID NO: 11 - protein, 120 aa, Lama glama
EVKLQQSGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRANWGQGTQVTVSSEPQ

SEQ ID NO: 12 - protein, 120 aa, Lama glama; identical to SEQ ID NO: 5

SEQ ID NO: 13 - protein, 120 aa, Lama glama; identical to SEQ ID NO: 5

SEQ ID NO: 14 - protein, 118 aa, Lama glama
EVQLQESGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 15 - protein, 122 aa, Lama glama
EVKLQQSGGDLVQAGGSLRLSCAASGRTPYVMGWFRQARGKEREFVAAITSSFTRYLADSVKGRFAISRDNAKNTVYLQMNSLQPEDTAVYYCAAGSIVRPSTDAYDYWGQGTQVTVSSEAQ

SEQ ID NO: 16 - protein, 118 aa, Lama glama; identical to SEQ ID NO: 2

SEQ ID NO: 17 - protein, 127 aa, Lama glama
EVQLQESGGGLVQAGDSLRLSCLVSGRSFNSYTMGWFRQAPGKEREFVAAILWSGPTTYYADSVKGRFTISRDNAKNTVYLQMNSLKPEDTAVYYCAAALGVLVLAPGNVYSYWGQGTQVTVSSAHH

SEQ ID NO: 18 - protein, 120 aa, Lama glama; identical to SEQ ID NO: 5

SEQ ID NO: 19 - protein, 120 aa, Lama glama
EVKLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTARGNWCQGTQVTVSSEPQ

SEQ ID NO: 20 - protein, 120 aa, Lama glama; identical to SEQ ID NO: 6

SEQ ID NO: 21 - protein, 120 aa, Lama glama
QVKLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTRVTVSSEPQ

SEQ ID NO: 22 - protein, 120 aa, Lama glama; identical to SEQ ID NO: 6

SEQ ID NO: 23 - protein, 120 aa, Lama glama
EVKLQESGGGLVQSGGSLRLSCIASGSTVSDNTMGWYRQAPGKQRELVTVISSVGVTNVAQSVKGRFTISRDNAKNTVTVYLQMSSLKPEDTAVYYCNALGTGRANWGQGTQVTVSSEPQ

SEQ ID NO: 24 - protein, 120 aa, Lama glama; identical to SEQ ID NO: 5

SEQ ID NO: 25 - protein, 126 aa, Lama glama
EVKLQQSGGGLVQPGKSLRLSCEASARAAGVGWFRQAPGLEREYVGSFLWNTGRTHYPESLKDRFTISKDNAKNTVYLQINSLKPEDTAIYYCAAVQLPIRTSLTEPATYTFWGQGTQVTVASEPQ

SEQ ID NO: 26 - protein, 126 aa, Lama glama; identical to SEQ ID NO: 7

SEQ ID NO: 27 - protein, 127 aa, Lama glama
QVQLQESGGGLVQAGDSLRLSCAGSGRAFRTYAMGWFRQAPGKEREFVARMTFGGGDTDYAGSVKGRFTISKDYAKNILYLQMNSLNPEDTAVYYCAADRTYRDLLQSRTVDYWGQGTQVTVSSAHH

SEQ ID NO: 28 - protein, 120 aa, Lama glama
EVQLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGQGNWGQGTQVTVSSEPQ

SEQ ID NO: 29 - protein, 120 aa, Lama glama
EVKLQQSGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTQVTVSSEPQ

SEQ ID NO: 30 - protein, 122 aa, Lama glama
QVKLQESGGDLVQAGGSLRLSCAASGRTPYVMGWFRQARGKEREFVAAITSSFTRYLADSVKGRFAISRDNAKNTVYLQMNSLQPEDTAVYYCAAGSIVRPSTDAYDYWGQGTQVTVSSEPQ

SEQ ID NO: 31 - protein, 118 aa, Lama glama
QVKLQESGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 32 - protein, 118 aa, Lama glama
QVKLQESGGGLVQSGGSLRLSCIASGSTVSGNTMGWYRQAPGKQHELVAVISSVGVSNVAQSVKGRFAISRDNAKNTVYLQMSSLKPEDTAVYYCNALGTGRANWGQGTQVTVSSEPQ

SEQ ID NO: 33 - protein, 118 aa, Lama glama
QVQLQESGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 34 - protein, 120 aa, Lama glama
EVKLQESGGGLVQSGGSLRLSCIASGSTVSDNTMGWYRQAPGKQRELVAVISSVGVANVAQSVKGRFTISRDNAKNTVTVYLQMSSLKPEDTAVYYCNALGTGRGNWGQGTQVTVSSEPQ

SEQ ID NO: 35 - protein, 118 aa, Lama glama
EVKLQQSGGGLVQSGGSLRLSCIASGSTVSGNTMGWYRQAPGKQRELVAVISSVGVSNVAQSVKGRFAISRDNAKNTVYLQMSSLKPEDTAVYYCNALGTGRANWGQGTQVTVSSEPQ

SEQ ID NO: 36 - protein, 120 aa, Lama glama
EVKLQESGGGLVQSGGSLRLSCIASGSTASDNTMGWYRQAPGKQRELVAVISSVGVTNVAQSVKGRFTISRDNAKNTVTAYLQMSSLKPEDTAVYYCNALGTGRANWGQGTQVTVSSEPQ

SEQ ID NO: 37 - protein, 120 aa, Lama glama
QVKLHESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTQVTVSSEPQ

SEQ ID NO: 38 - protein, 120 aa, Lama glama
EVQLQQSGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRANWGQGTQVTVSSEPQ

SEQ ID NO: 39 - protein, 120 aa, Lama glama; identical to SEQ ID NO: 6

SEQ ID NO: 40 - protein, 120 aa, Lama glama; identical to SEQ ID NO: 4

SEQ ID NO: 41 - protein, 120 aa, Lama glama
EVQLQESGGGLVQSGGSLRLSCIASGSTVSDNTMGWYRQAPGKQRELVAVISSVGVTNVAQSVKGRFTISRDNAKNTVTVYLQMSSLKPEDTAVYYCNALGTGRGNWGQGTQVTVSSEPQ

SEQ ID NO: 42 - protein, 118 aa, Lama glama; identical to SEQ ID NO: 2

SEQ ID NO: 43 - protein, 118 aa, Lama glama
EVKLQESGGGLVQSGGSLRLSCIASGSTVSGNTMGWYRQAPGKQRELVAVISSVGVSNVAQSVKGRFAISRDNAKNTVYLQMSSLKPEDTDVYYCNALGTGRANWGQGTQVTVSSEPQ

SEQ ID NO: 44 - protein, 120 aa, Lama glama; identical to SEQ ID NO: 6

SEQ ID NO: 45 - protein, 118 aa, Lama glama
EVQLQQSGGGLVQSGGSLRLSCIASGSTVSGNTMGWYRQAPGKQRELVAVISSVGVSNVAQSVKGRFAISRDNAKSTVYLQMSSLKPEDTAVYYCNALGTGRANWGQGTQVTVSSEPQ

SEQ ID NO: 46 - protein, 118 aa, Lama glama
QVKLQQSGGGLVQSGGSLRLSCIASGNTVSGNTMGWYRQAPGKQRELVAVISSVGVSNVAQSVKGRFAISRDNAKNTVYLQMSSLKPEDTAVYYCNALGTGRANWGQGTQVTVSSGPQ

SEQ ID NO: 47 - protein, 114 aa, Artificial Sequence
  Random Phage used as Negative Control
EVQLQESGGGLVQAGGSLRLSCAASGSTFSNEPFGWYRGAPGKLRELVGTISAGGNTNYVDAVKGRFMISRDNGRRMVYLQMNNLRPDDTAVYYCWNRGYWGQGTQVTVSSEPQ

SEQ ID NO: 48 - protein, 121 aa, Artificial Sequence
  Random Phage used as Negative Control
KVKLQESGGGEVQPGGSLRLSCAASGFNLESSAIGWFRQAPGSEREEVSCISTKGNIKDTPSVRGRFTVSRDNVKNIVYLQMNNLKPEDTAVYYCATSCWQHIDGILWGQGTQVTVSSEPQ

SEQ ID NO: 49 - protein, 36 aa, Artificial Sequence
  6-His and Myc tag
TPKPQPQPQPAAAHHHHHHGAAEQKLISEEDLNGAA

SEQ ID NO: 50 - protein, 32 aa, Artificial Sequence
  6-His and Myc tags
SEDPSSAAAHHHHHHGAAEQKLISEEDLNGAA

SEQ ID NO: 51 - DNA, 57 bp, Artificial Sequence
  primer
catgccatgactcgcggcccagccggccatggccsaggtsmarctgcagsagtcwgg

SEQ ID NO: 52 - DNA, 50 bp, Artificial Sequence
  primer
ttttccttttgcggccgcggccgcggagctggggtcttcgctgtggtgcg

SEQ ID NO: 53 - DNA, 57 bp, Artificial Sequence
  primer
ggattgggttgcggccgctggttgtggttgtggttgtggttttggtgtctggggttc

(19) United States
(12) Patent Application Publication
(10) Pub. No.: US 2011/0200525 A1
(43) Pub. Date: Aug. 18, 2011

(54) VHH ANTIBODY FRAGMENTS FOR USE IN THE DETECTION AND TREATMENT OF CANCER

(76) Inventors: Edward F. Patz, JR., Chapel Hill, NC (US); Elizabeth B. Gottlin, Chapel Hill, NC (US); Michael J. Campa, Durham, NC (US)

(21) Appl. No.: 13/120,511

(22) PCT Filed: Oct. 9, 2009
(86) PCT No.: PCT/US09/60159

§ 371 Date: Apr. 27, 2011

(30) Foreign Application Priority Data
Oct. 9, 2008 (US) 61/104202

(51) Int. Cl. A61K 51/00; G01N 33/574; G01N 33/563; C07K 16/18; A61K 49/00; A61K 39/395; A61K 49/06; A61K 49/22; A61K 9/127; A61P 35/00; G01N 21/64; A61P 35/04; G01N 23/00

(52) U.S. Cl. 424/1.11; 435/7.23; 530/387.7; 530/387.3; 424/9.1; 424/138.1; 424/9.6; 424/9.3; 424/9.5; 424/450; 424/133.1

(57) ABSTRACT

The presently disclosed subject matter provides ligands for detecting and imaging cancer cells and tumors, and for guided delivery of an active agent to cancer cells and tumors. In some embodiments the ligands comprise an antibody fragment, wherein the antibody fragment comprises a VHH domain. In some embodiments a composition is provided for targeting of cancer cells or tumors. Also provided are methods for delivery of a composition to a target tissue or tumor in a subject. Also provided are methods for imaging a target tissue or tumor in a subject. In some embodiments methods for treating a tumor in a subject are provided. Also provided are methods for diagnosing a tumor in a subject.

### CROSS-REFERENCE TO RELATED APPLICATIONS

This application claims benefit of U.S. Provisional Patent Application Ser. No. 61/104,202, titled “VHH Antibody Fragments for Use in the Detection and Treatment of Cancer,” filed Oct. 9, 2008, which is herein incorporated by reference in its entirety.

### TECHNICAL FIELD

The presently disclosed subject matter generally relates to ligands for detecting and imaging cancer cells and tumors. Also provided are ligands for guided delivery of an active agent. Also provided are therapeutic and diagnostic uses for the same.

### BACKGROUND

Cancer continues to be a significant worldwide public health issue. More effective approaches for detecting and treating cancer continue to be pursued.

Taking the example of lung cancer, although advances in noninvasive imaging have improved the ability to detect lung cancer, >75% of lung cancer patients present with advanced stage disease when therapeutic options are limited (Mountain, C. F. Revisions in the International System for Staging Lung Cancer. Chest 111:1710-1717, 1997). Even those patients who present with clinical stage I lung cancer have at best a 60% 5-year survival rate, signifying that a large percentage of all stage I patients have undetectable metastatic disease at the time of presentation. (Mountain, C. F. Revisions in the International System for Staging Lung Cancer. Chest 111:1710-1717, 1997). These statistics underscore the need for improvements in early detection strategies.

Additionally, lung cancer accounts for more cancer deaths than any other malignancy. Despite advances in diagnostic capabilities and treatment, lung cancer mortality has not significantly changed over the past several decades. Most patients present with inoperable disease when therapeutic options including chemotherapy and radiotherapy are rarely curative.

Accordingly, there remains an unmet need for approaches that provide for the detection and treatment of cancer, including but not limited to lung cancer.

### SUMMARY

In some embodiments the presently disclosed subject matter provides a composition for targeting of cancer cells, wherein the composition comprises one or more targeting ligands comprising an antibody fragment, wherein the antibody fragment comprises a VHH domain comprising a sequence as set forth in SEQ ID NOs.: 1-46, or a variant or a derivative thereof. In some embodiments the antibody fragment, or variant thereof, is humanized. In some embodiments the one or more targeting ligands bind to one or more tumor types selected from among bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head, a neck tumor, and a solid tumor.

In some embodiments the composition further comprises a detectable label, a therapeutic agent, a carrier, or combinations thereof. In some embodiments the detectable label is an in vivo detectable label, which optionally can be detected using magnetic resonance imaging, scintigraphic imaging, ultrasound, or fluorescence. In some embodiments the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹ rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

In some embodiments the therapeutic agent is selected from the group consisting of a radionuclide, a cytotoxin, and a chemotherapeutic agent. In some embodiments the carrier is selected from the group consisting of a liposome, a microcapsule, and combinations thereof. In some embodiments the targeting ligand itself acts as a therapeutic agent.

In some embodiments the presently disclosed subject matter provides a method for delivery of a composition to a target tissue in a subject, the method comprising: administering to the subject a therapeutic composition, a diagnostic composition, or a combination thereof, wherein the therapeutic composition, diagnostic composition, or combination thereof, comprises one or more targeting ligands comprising an antibody fragment, wherein the antibody fragment comprises a VHH domain comprising a sequence as set forth in SEQ ID NOs.: 1-46, or a variant or a derivative thereof, whereby the composition is selectively targeted to the target tissue. In some embodiments the antibody fragment, or variant thereof, is humanized. In some embodiments

In some embodiments the composition further comprises a detectable label, a therapeutic agent, a carrier, or combinations thereof. In some embodiments the detectable label is an in vivo detectable label, which optionally can be detected using magnetic resonance imaging, scintigraphic imaging, ultrasound, or fluorescence. In some embodiments the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁶⁵rhenium, ¹⁰¹ rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

In some embodiments the therapeutic agent is selected from the group consisting of a radionuclide, a cytotoxin, and a chemotherapeutic agent. In some embodiments the targeting ligand itself acts as a therapeutic agent. In some embodiments the carrier is selected from the group consisting of a liposome, a microcapsule, and combinations thereof.

In some embodiments the target tissue comprises a tumor. In some embodiments the tumor is a primary or a metastasized tumor. In some embodiments the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments the subject is a warm-blooded vertebrate.

In some embodiments the presently disclosed subject matter provides a method for imaging a target tissue in a subject, the method comprising: administering to the subject a composition comprising one or more targeting ligands comprising an antibody fragment, wherein the antibody fragment comprises a VHH domain comprising a sequence as set forth in SEQ ID NOs.: 1-46, or a variant or a derivative thereof, wherein the composition further comprises an in vivo detectable label; and detecting the composition. In some embodiments the antibody fragment, or variant thereof, is humanized.

In some embodiments the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium. In some embodiments detecting the composition comprises detecting the in vivo detectable label using magnetic resonance imaging, scintigraphic imaging, ultrasound, or fluorescence.

In some embodiments the composition further comprises a therapeutic agent, a carrier, or combinations thereof. In some embodiments the therapeutic agent is selected from the group consisting of a radionuclide, a cytotoxin, and a chemotherapeutic agent. In some embodiments the targeting ligand itself acts as a therapeutic agent. In some embodiments the carrier is selected from the group consisting of a liposome, a microcapsule, and combinations thereof.

In some embodiments the target tissue comprises a tumor. In some embodiments the tumor is a primary or a metastasized tumor. In some embodiments the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments the subject is a warm-blooded vertebrate.

The presently disclosed subject matter further provides a method for treating a tumor in a subject, the method comprising: providing a subject with a tumor; and administering to the subject a therapeutic composition comprising one or more targeting ligands comprising an antibody fragment, wherein the antibody fragment comprises a VHH domain comprising a sequence as set forth in SEQ ID NOs.: 1-46, or a variant or a derivative thereof. In some embodiments the antibody fragment, or variant thereof, is humanized.

In some embodiments the composition further comprises a therapeutic agent, detectable label, a carrier, or combinations thereof. In some embodiments the detectable label is an in vivo detectable label, which optionally can be detected using magnetic resonance imaging, scintigraphic imaging, ultrasound, or fluorescence. In some embodiments the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

In some embodiments the carrier is selected from the group consisting of a liposome, a microcapsule, and combinations thereof. In some embodiments the therapeutic agent is selected from the group consisting of a radionuclide, a cytotoxin, and a chemotherapeutic agent. In some embodiments the targeting ligand itself acts as a therapeutic agent.

In some embodiments the tumor is a primary or a metastasized tumor. In some embodiments the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments the tumor over-expresses epidermal growth factor receptor (EGFR). In some embodiments the subject is a warm-blooded vertebrate.

In some embodiments the presently disclosed subject matter provides a method for determining the presence of a tumor, the method comprising: biopsying a suspected tumor; contacting the biopsy of the suspected tumor with a composition comprising an antibody fragment, wherein the antibody fragment comprises a VHH domain comprising a sequence as set forth in SEQ ID NOs.: 1-46, or a variant or a derivative thereof, wherein the composition further comprises a detectable label; and detecting the composition bound to the biopsy of the suspected tumor, whereby the detection of the composition on the biopsy of the suspected tumor determines that the suspected tumor is a tumor.

In some embodiments the antibody fragment, or variant thereof, is humanized. In some embodiments the composition further comprises a carrier selected from the group consisting of a liposome, a microcapsule, and combinations thereof.

In some embodiments the detectable label is a fluorescent or radioactive label. In some embodiments the detection of the composition comprises detecting the detectable label using autoradiography or fluorescence. In some embodiments the method further comprises rinsing the biopsy of the suspected tumor to remove unbound targeting ligands from the biopsy of the suspected tumor.

In some embodiments the tumor is a primary or a metastasized tumor. In some embodiments the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments the tumor over-expresses epidermal growth factor receptor (EGFR). In some embodiments the biopsy is obtained from a warm-blooded vertebrate.

It is an object of the presently disclosed subject matter to provide novel targeting ligands, and/or therapeutic and/or diagnostic methods using the same. This and others objects are achieved in whole or in part by the presently disclosed subject matter.

An object of the presently disclosed subject matter having been stated above, other objects and advantages of the presently disclosed subject matter will become apparent to those skilled in the art after a study of the following description and Examples.

### BRIEF DESCRIPTION OF THE DRAWINGS

FIG. 1 is a schematic representation of the phagemid vector pHEN1 modified to contain a carboxyl terminal 6-His tag (pHEN1-H6) and including a VHH-encoding insert upstream of the bacteriophage fd gene III. The pHEN1-H6 replicates as a plasmid in E. coli and in some embodiments of the presently disclosed subject matter was used to generate a phagemid-VHH library.

FIG. 2A is a schematic key to the photographic images (FIGS. 2B-2D) of 96-well plates employed in a cell-based enzyme-linked immunosorbent assay (ELISA). One set of 94 “rescued” phage was transferred to each of 3 plates containing parental (NR6V; FIG. 2B), wild type EGFR-expressing (NR6W; FIG. 2C), or EGFRvIII-expressing (NR6M; FIG. 2D) cells. Phage binding was detected with an anti-phage horseradish peroxidase (HRP) conjugate and a peroxidase substrate.

FIG. 3 is a plot of antibody competition of phage binding in cell-based ELISA. Antibodies were serially diluted and placed in wells of a 96-well microplate containing fixed cells, along with a constant amount of phage. After incubation of cells, antibodies and phage for 1 hr, the plates were washed and remaining phage were quantitated with an anti-phage-HRP conjugate at an absorbance of 405 nm. Antibody concentration is expressed as nM. Actin monoclonal antibody (mAb) MS-1295 served as a control and is represented by a circle. Experimental EGFR mAbs are as follows: B10D11, triangle; L8A4, inverted triangle; H11, square; and F2A2, diamond.

FIGS. 4A and 4B identify the target of VHH domains as EGFR. FIG. 4A is an autoradiograh of an anti-EGFR Western blot used to identify the targets of VHH domains as EGFR. The samples run on the Western blot are proteins pulled down by and eluted from VHH domains immobilized on a nickel chelate plate. FIG. 4B is a MS/MS sequencing report of protein pulled down by and eluted from VHH122 immobilized on Sepharose beads. The superscripts in the MS/MS sequencing report refer to the following: ¹Medical Research Council database; ²Number of peptides that match the theoretical digest of the primary protein identified; ³Score of the quality of the peptide-mass fingerprint match and the quality of the MS/MS peptide fragment ion matches; ⁴Score of the quality of MS/MS peptide fragment ion matches only; and ⁵Significance of MS & MS/MS Score: probability that observed score is due to chance (Expect Values of 0.05 or less indicate a significant hit).

FIG. 5 is a histogram of flow cytometry analysis showing the specificity of VHH122 for EGFR-expressing cells. Cells expressing (NR6W) or not expressing (NR6V) EGFR were incubated with VHH122 or control VHH RP2, stained with anti-His₆ fluorescein isothiocyanate (FITC)-conjugated antibody and analyzed in a flow cytometer.

FIG. 6 is a guide tree schematic of 15 EGFR-specific VHH domains utilizing the Neighbor Joining algorithm of Saitou and Nei (Saitou, N. & Nei, M. (1987) Mol Biol Evol 4:406-425).

FIG. 7 is a clustal W alignment of EGFR-specific VHH domains in Groups 1 and 2 of the guide tree of FIG. 6. Residues with dark grey shading are identical; residues with light grey shading are most prevalent within the group. All amino acid sequences are derived from double-stranded sequence analysis of each cloned insert. The VHH domains in FIG. 7 correspond to the sequences provided herein as follows: VHH102, SEQ ID NO: 8; VHH104, SEQ ID NO: 9; VHH134, SEQ ID NO: 23; VHH110, SEQ ID NO: 12; VHH130, SEQ ID NO: 21; VHH114, SEQ ID NO: 14; VHH215, SEQ ID NO: 33; VHH245, SEQ ID NO: 45; VHH219, SEQ ID NO: 35; VHH255, SEQ ID NO: 46; VHH03, SEQ ID NO: 1; VHH139, SEQ ID NO: 25; VHH107, SEQ ID NO: 11; VHH122, SEQ ID NO: 17; VHH205, SEQ ID NO: 31.

FIGS. 8A and 8B are autoradiographs of Western blots illustrating VHH122 stability in serum. VHH122 was added at a concentration of 10 ug/ml to either mouse serum (FIG. 8A) or normal human serum (FIG. 8B) and the mixtures were incubated at 37° C. At time points of 0, 0.5, 1, 2 and 4 hours-post incubation aliquots were removed and heated in SDS-Protein Sample Buffer. Samples were subjected to SDS-PAGE, electrophoretically transferred to nitrocellulose, and the Western blots probed with an anti-myc HRP conjugate to detect the myc-tagged VHH. VHH122 appeared to be stable over the course of >4 hr in both mouse and human sera.

FIG. 9 is a scatter plot depicting the saturation binding of ¹²⁵I-labelled VH122 on an EGFR-expressing cell. The calculated dissociation constant (Kd) of VH122 is 46 nM.

FIG. 10 is an autoradiograph of a Western blot of protein lysates from A431 and 5M2 cells probed with mAbs to EGFR and actin. Lanes 1-4 contain 2.5, 5, 10 and 15 ug protein, respectively.

FIGS. 11A-11F are MicroPET images of tumor-bearing mice injected with ¹²⁴I-labeled VHH domains. Labeled probe (35-50 uCi) was injected via the tail vein and 15-minute images were acquired approximately 45 minutes after injection. The figures are labeled with “T”, “K” and “B”, where T=tumor; K=kidney; and B=bladder.

### BRIEF SUMMARY OF THE SEQUENCE LISTING

SEQ ID NOs.: 1-46 are polypeptide sequences of VHH domain proteins capable of binding Epidermal Growth Factor Receptor (EGFR). Further details of the VHH domains of SEQ ID NOs. 1-46 are set forth in Table 1 and throughout the instant disclosure.

SEQ ID NOs.: 47 and 48 are polypeptide sequences of random phage domains used as negative controls. Further details are set forth in Table 1.

SEQ ID NOs.: 49 and 50 are sequences of amino acids comprising 6-His and Myc tags that can be included as part of the VHH domains and random phage domains of the presently disclosed subject matter.

SEQ ID NO.: 51 is a reverse primer used in the polymerase chain reaction (PCR) amplification of heavy chain-only llama IgG for the construction of the VHH domain library disclosed herein.

SEQ ID NOs.: 52 and 53 are forward primers used in the PCR amplification of heavy chain-only llama IgG for the construction of the VHH domain library disclosed herein.

### DETAILED DESCRIPTION

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements may be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “cell” refers not only to the particular subject cell (e.g., a living biological cell), but also to the progeny or potential progeny of such a cell. Because certain modifications can occur in succeeding generations due to either mutation or environmental influences, such progeny might not, in fact, be identical to the parent cell, but are still included within the scope of the term as used herein.

The term “ligand” as used herein refers to a molecule or other chemical entity having a capacity for binding to a target. A ligand can comprise a peptide, an oligomer, a nucleic acid (e.g., an aptamer), a small molecule (e.g., a chemical compound), an antibody or fragment thereof, a nucleic acid-protein fusion, and/or any other affinity agent.

The term “small molecule” as used herein refers to a compound, for example an organic compound, with a molecular weight in some embodiments of less than about 1,000 daltons, in some embodiments less than about 750 daltons, in some embodiments less than about 600 daltons, and in some embodiments less than about 500 daltons. A small molecule also has a computed log octanol-water partition coefficient in some embodiments in the range of about −4 to about +14, and in some embodiments in the range of about −2 to about +7.5.

The term “target tissue” as used herein refers to an intended site for accumulation of a ligand following administration to a subject. For example, the methods disclosed herein can employ a target tissue comprising a tumor or cancerous tissues.

The term “control tissue” as used herein refers to a site suspected to substantially lack binding and/or accumulation of an administered ligand. For example, in accordance with the methods of the presently disclosed subject matter, a non-cancerous tissue can be a control tissue.

The terms “target” or “target molecule” as used herein each refer to any substance that is selectively bound by a ligand. Thus, the term “target molecule” encompasses macromolecules including but not limited to proteins (e.g., receptors), nucleic acids, carbohydrates, lipids, and complexes thereof.

The terms “targeting” or “homing”, as used herein to describe the in vivo activity of a ligand following administration to a subject, each refer to the preferential movement and/or accumulation of a ligand in a target tissue as compared with a control tissue.

The terms “selective targeting” or “selective homing” as used herein each refer to a preferential localization of a ligand that results in an amount of ligand in a target tissue that is in some embodiments about 2-fold greater than an amount of ligand in a control tissue, in some embodiments about 5-fold or greater than an amount of ligand in a control tissue, and in some embodiments an amount that is about 10-fold or greater than an amount of ligand in a control tissue. The terms “selective targeting” and “selective homing” also refer to binding or accumulation of a ligand in a target tissue concomitant with an absence of targeting to a control tissue, in some embodiments the absence of targeting to all control tissues.

The term “absence of targeting” is used herein to describe substantially no binding or accumulation of a ligand in all control tissues where an amount of ligand is detectable.

The terms “targeting ligand”, “targeting molecule”, “homing ligand”, and “homing molecule” as used herein each refer to a ligand that displays targeting activity. In some embodiments, a targeting ligand displays selective targeting.

The term “binding” refers to an affinity between two molecules, for example, a ligand and a target molecule. As used herein, “binding” means a preferential binding of one molecule for another in a mixture of molecules. In some embodiments, the binding of a ligand to a target molecule can be considered specific or selective if the binding affinity is in some embodiments about 1×10⁴ M⁻¹ to about 1×10⁶ M⁻¹ or greater.

The phrase “specifically (or selectively) binds”, when referring to the binding capacity of a ligand, refers to a binding reaction which is determinative of the presence of the target in a heterogeneous population of other biological materials. The phrase “specifically (or selectively) binds” also refers to selectively targeting, as defined hereinabove.

The phases “substantially lack binding” or “substantially no binding”, as used herein to describe binding of a ligand in a control tissue, refers to a level of binding that encompasses non-specific or background binding, but does not include specific binding.

The terms “humanized” or “humanized antibody”, as used herein, refers to an antibody derived from a non-human antibody, for example but not limited to murine, that retains or substantially retains the antigen-binding properties of the parent antibody but which is less immunogenic in humans than a non-humanized antibody.

The term “tumor” as used herein refers to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The term “tumor” also encompasses solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas.

The term “subject” as used herein refers to any invertebrate or vertebrate species. The methods and compositions disclosed herein are particularly useful in the treatment and diagnosis of warm-blooded vertebrates. Thus, the presently disclosed subject matter concerns mammals and birds. More particularly provided is the treatment and/or diagnosis of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

### II. Targeting Compositions

Lung cancer is the leading cause of death from cancer in the United States of America and the world. By the time of diagnosis, most tumors have already metastasized. Disclosed herein, in accordance with some embodiments of the presently disclosed subject matter, are antibody fragments derived from llama antibodies that are specific to a lung tumor cell marker, epidermal growth factor receptor (EGFR). Though expressed in numerous normal tissues, EGFR is over-expressed in a number of cancers and tumors, including lung cancer. These antibody fragments, termed VHH domains or EGFR-specific VHH domains, have the specificity and affinity of full-length antibodies. Further, because they are only one-tenth the size of a full-length antibody, they are predicted to be able to infiltrate tumors substantially faster.

Thus, the presently disclosed subject matter pertains in some embodiments to the development and use of VHH antibody fragments to detect EGFR on lung cancer cells and thus diagnose lung cancer at an earlier more curable phase. Additionally, in some embodiments, the presently disclosed VHH molecules can be used to identify and diagnose other types of cancer cells or tumors, particularly those that over-express EGFR, by targeting EGFR. Further, in some embodiments of the presently disclosed subject matter, the VHH molecules can be used as targeting ligands to which a toxin can be attached, as necessary, to kill tumor cells. In some embodiments, a VHH molecule itself can be a toxic agent. Further, in some embodiments of the presently disclosed subject matter, targeting ligands such as VHH can be used to visualize and image tumors and cancer cells.

The presently disclosed subject matter includes a study of the targeting activity of VHH antibodies in tumor-bearing subjects. See the Examples below. By way of example and not limitation, VHH domains of the presently disclosed subject matter are included in Table 1 and the sequence listing. In the peptide sequences of VHH domains set forth in Table 1 the signal sequences have been removed. All the peptide sequences have been derived from single-stranded DNA sequencing except where noted as double-stranded sequence.

TABLE 1
Translated sequences of mature free VHH domain
proteins selected on Epidermal Growth Factor
Receptor (EGFR)
VHH03 (SEQ ID NO: 1) (Double-stranded sequence
confirmed; contains stop codon at position 13
(would be Q in supE strain of E. coli); in phage,
this VHH domain recognizes wild type, not VIII
mutant, EGFR)
EVKLQQSGGGLVQPGGSLTLSCVTSGFTFSTYGMDWVRQAPGKGFE
WVASISFGGGITNYGDFVKGRFTISRDNAKNTLYLHMNGLKPDDTAVY
YCQMGSKRGQGTQVTVSSEPQ
VHH13 (SEQ ID NO: 2)
EVKLQESGGGLVQSGGSLRLSCIASGSTVSGNTMGWYRQAPGKQREL
VAVISSVGVSNVAQSVKGRFAISRDNAKNTVYLQMSSLKPEDTAVYYC
NALGTGRANWGQGTQVTVSSEPQ
VHH19 (SEQ ID NO: 3)
QVKLQQSGGGLVQSGGSLRLSCIASGSTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
VHH20 (SEQ ID NO: 4)
EVQLQQSGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRGNWGQGTQVTVSSEPQ
VHH24 (SEQ ID NO: 5)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRGNWGQGTQVTVSSEPQ
VHH97 (SEQ ID NO: 6)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH101 (SEQ ID NO: 7)
EVKLQESGGGLVQPGKSLRLSCEASARAAGVGWFRQAPGLEREYVG
SFLWNTGRTHYPESLKDRFTISKDNAKNTVYLQINSLKPEDTAIYYCA
AVQLPIRTSLTEPATYTFWGQGTQVTVASEPQ
VHH102 (SEQ ID NO: 8) (Double-stranded sequence
confirmed)
EVKLQESGGGLVQSGGSLRLSCIASGSTVSGNTMGWYRQAPGKQREL
VAVISSVGVSNVAQSVKGRFAISRDNAKNTVYLQMSSLKPEDTAVYYC
NALGTGRANWGQGTQVTVSSEPQ
VHH104 (SEQ ID NO: 9) (Double-stranded sequence
confirmed)
EVQLQQSGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRGNWGQGTQVTVSSEPQ
VHH105 (SEQ ID NO: 10)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH107 (SEQ ID NO: 11) (Double-stranded sequence
confirmed)
EVKLQQSGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH110 (SEQ ID NO: 12) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRGNWGQGTQVTVSSEPQ
VHH111 (SEQ ID NO: 13) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRGNWGQGTQVTVSSEPQ
VHH114 (SEQ ID NO: 14) (Double-stranded sequence
confirmed)
EVQLQESGGGLVQSGGSLRLSCIASGSTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
VHH115 (SEQ ID NO: 15) (Double-stranded sequence
confirmed)
EVKLQQSGGDLVQAGGSLRLSCAASGRTPYVMGWFRQARGKEREFV
AAITSSFTRYLADSVKGRFAISRDNAKNTVYLQMNSLQPEDTAVYYCA
AGSIVRPSTDAYDYWGQGTQVTVSSEAQ
VHH116 (SEQ ID NO: 16) (ends with PH instead of
AA; also seen in some random library phage)
EVKLQESGGGLVQSGGSLRLSCIASGSTVSGNTMGWYRQAPGKQREL
VAVISSVGVSNVAQSVKGRFAISRDNAKNTVYLQMSSLKPEDTAVYYC
NALGTGRANWGQGTQVTVSSEPQ
VHH122 (SEQ ID NO: 17) (Double-stranded sequence
confirmed)
EVQLQESGGGLVQAGDSLRLSCLVSGRSFNSYTMGWFRQAPGKERE
FVAAILWSGPTTYYADSVKGRFTISRDNAKNTVYLQMNSLKPEDTAVY
YCAAALGVLVLAPGNVYSYWGQGTQVTVSSAHH
VHH124 (SEQ ID NO: 18) (Double-stranded sequence
confirmed)
EVKLQESGGGLVQSGGSLRLSCIASGSTVSDNTMGWYRQAPGKQRELV
AVISSVGVTNVAQSVKGRFTISRDNAKNTVTVYLQMSSLKPEDTAVYY
CNALGTGRGNWGQGTQVTVSSEPQ
VHH127 (SEQ ID NO: 19) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTARGNWCQGTQVTVSSEPQ
VHH128 (SEQ ID NO: 20) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH130 (SEQ ID NO: 21) (Double-stranded sequence
confirmed)
QVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRGNWGQGTRVTVSSEPQ
VHH132 (SEQ ID NO: 22) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH134 (SEQ ID NO: 23) (Double-stranded sequence
confirmed)
EVKLQESGGGLVQSGGSLRLSCIASGSTVSDNTMGWYRQAPGKQREL
VTVISSVGVTNVAQSVKGRFTISRDNAKNTVTVYLQMSSLKPEDTAVY
YCNALGTGRANWGQGTQVTVSSEPQ
VHH137 (SEQ ID NO: 24) (Double-stranded sequence
confirmed)
EVKLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRANWGQGTQVTVSSEPQ
VHH139 (SEQ ID NO: 25) (Double-stranded sequence
confirmed)
EVKLQQSGGGLVQPGKSLRLSCEASARAAGVGWFRQAPGLEREYVG
SFLWNTGRTHYPESLKDRFTISKDNAKNTVYLQINSLKPEDTAIYYCA
AVQLPIRTSLTEPATYTFWGQGTQVTVASEPQ
VHH141 (SEQ ID NO: 26) (Double-stranded sequence
confirmed)
EVKLQESGGGLVQPGKSLRLSCEASARAAGVGWFRQAPGLEREYVG
SFLWNTGRTHYPESLKDRFTISKDNAKNTVYLQINSLKPEDTAIYYCA
AVQLPIRTSLTEPATYTFWGQGTQVTVASEPQ
VHH142 (SEQ ID NO: 27) (Double-stranded sequence
confirmed)
QVQLQESGGGLVQAGDSLRLSCAGSGRAFRTYAMGWFRQAPGKERE
FVARMTFGGGDTDYAGSVKGRFTISKDYAKNILYLQMNSLNPEDTAVY
YCAADRTYRDLLQSRTVDYWGQGTQVTVSSAHH
VHH201 (SEQ ID NO: 28) (Double-stranded sequence
confirmed)
EVQLQESGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGQGNWGQGTQVTVSSEPQ
VHH202 (SEQ ID NO: 29) (Double-stranded sequence
confirmed)
EVKLQQSGGGLVQSGGSLRLSCIASGSTVSDNTMGWYRQAPGKQREL
VAVISSVGVTNVAQSVKGRFTISRDNAKNTVTVYLQMSSLKPEDTAVY
YCNALGTGRGNWGQGTQVTVSSEPQ
VHH203 (SEQ ID NO: 30) (contains ambiguous stop
codon or Q after signal sequence (would be Q in
supE strain of E. coli so amino acid 1 = Q))
QVKLQESGGDLVQAGGSLRLSCAASGRTPYVMGWFRQARGKEREFV
AAITSSFTRYLADSVKGRFAISRDNAKNTVYLQMNSLQPEDTAVYYCA
AGSIVRPSTDAYDYWGQGTQVTVSSEPQ
VHH205 (SEQ ID NO: 31) (Double-stranded sequence
confirmed; contains ambiguous stop codon or Q
after signal sequence (would be Q in supE
strain of E. coli so amino acid 1 = Q))
QVKLQESGGGLVQSGGSLRLSCIASGSTVSGNTMGWYRQAPGKQRE
LVAVISSVGVSNVAQSVKGRFAISRDNAKNIVYLQMSSLKPEDTAVYY
CNALGTGRANWGQGTQVTVSSEPQ
VHH212 (SEQ ID NO: 32)
QVKLQESGGGLVQSGGSLRLSCIASGSTVSGNTMGWYRQAPGKQHE
LVAVISSVGVSNVAQSVKGRFAISRDNAKNTVYLQMSSLKPEDTAVYY
CNALGTGRANWGQGTQVTVSSEPQ
VHH215 (SEQ ID NO: 33) (Double-stranded sequence
confirmed)
QVQLQESGGGLVQSGGSLRLSCIASGSTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
VHH217 (SEQ ID NO: 34)
EVKLQESGGGLVQSGGSLRLSCIASGSTVSDNTMGWYRQAPGKQRE
LVAVISSVGVANVAQSVKGRFTISRDNAKNTVTVYLQMSSLKPEDTAVY
YCNALGTGRGNWGQGTQVTVSSEPQ
VHH219 (SEQ ID NO: 35) (Double-stranded sequence
confirmed)
EVKLQQSGGGLVQSGGSLRLSCIASGSTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
VHH221 (SEQ ID NO: 36)
EVKLQESGGGLVQSGGSLRLSCIASGSTASDNTMGWYRQAPGKQRE
LVAVISSVGVTNVAQSVKGRFTISRDNAKNTVTAYLQMSSLKPEDTAV
YYCNALGTGRANWGQGTQVTVSSEPQ
VHH222 (SEQ ID NO: 37)
QVKLHESGGGLVQSGGSLRLSCIASGSTVSDNTMGWYRQAPGKQRE
LVAVISSVGVINVAQSVKGRFTISRDNAKNIVTVYLQMSSLKPEDTAV
YYCNALGTGRGNWGQGTQVTVSSEPQ
VHH225 (SEQ ID NO: 38)
EVQLQQSGGGLVQSGGSLRLSCIASGSTVSDNTMGWYRQAPGKQRE
LVAVISSVGVINVAQSVKGRFTISRDNAKNTVTVYLQMSSLKPEDTAV
YYCNALGTGRANWGQGTQVTVSSEPQ
VHH229 (SEQ ID NO: 39)
EVKLQESGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRANWGQGTQVTVSSEPQ
VHH230 (SEQ ID NO: 40)
EVQLQQSGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRGNWGQGTQVTVSSEPQ
VHH231 (SEQ ID NO: 41)
EVQLQESGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRGNWGQGTQVTVSSEPQ
VHH232 (SEQ ID NO: 42)
EVKLQESGGGLVQSGGSLRLSCIASGSTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
VHH233 (SEQ ID NO: 43)
EVKLQESGGGLVQSGGSLRLSCIASGSTVSGNTMGWYRQAPGKQRE
LVAVISSVGVSNVAQSVKGRFAISRDNAKNTVYLQMSSLKPEDTDVYY
CNALGTGRANWGQGTQVTVSSEPQ
VHH235 (SEQ ID NO: 44)
EVKLQESGGGLVQSGGSLRLSCIASGSTVSDNTMGWYRQAPGKQRE
LVAVISSVGVTNVAQSVKGRFTISRDNAKNTVTVYLQMSSLKPEDTAV
YYCNALGTGRANWGQGTQVTVSSEPQ
VHH245 (SEQ ID NO: 45) (Double-stranded sequence
confirmed)
EVQLQQSGGGLVQSGGSLRLSCIASGSTVSGNTMGWYRQAPGKQRE
LVAVISSVGVSNVAQSVKGRFAISRDNAKSTVYLQMSSLKPEDTAVYY
CNALGTGRANWGQGTQVTVSSEPQ
VHH255 (SEQ ID NO: 46) (Double-stranded sequence
confirmed)
QVKLQQSGGGLVQSGGSLRLSCIASGNTVSGNTMGWYRQAPGKQRE
LVAVISSVGVSNVAQSVKGRFAISRDNAKNTVYLQMSSLKPEDTAVYY
CNALGTGRANWGQGTQVTVSSEPQ
Random Phages Used As Negative Controls
RP2 (SEQ ID NO: 47)
EVQLQESGGGLVQAGGSLRLSCAASGSTFSNEPFGWYRGAPGKLRE
LVGTISAGGNTNYVDAVKGRFMISRDNGRRMVYLQMNNLRPDDTAVYY
CWNRGYWGQGTQVTVSSEPQ
RP4 (SEQ ID NO: 48)
KVKLQESGGGEVQPGGSLRLSCAASGFNLESSAIGWFRQAPGSERE
EVSCISTKGNIKDTPSVRGRFTVSRDNVKNIVYLQMNNLKPEDTAVYY
CATSCWQHIDGILWGQGTQVTVSSEPQ
In some embodiments the VHH domains (i.e. SEQ ID NOs: 1-46) and random phages used as negative controls (i.e. SEQ ID NOs: 47-48) can further comprise 6-His and/or Myc tags. By way of example and not limitation the VHH domains and random phages of the presently disclosed subject matter can further comprise one or more of the following sequence of amino acids wherein the 6-His and Myc tags are in bold: TPKPQPQPQPAAAHHHHHHGAAEQKLISEEDLNGAA (SEQ ID NO: 49) SEDPSSAAAHHHHHHGAAEQKLISEEDLNGAA (SEQ ID NO: 50). For example, SEQ ID NO: 1 comprising the 6-His and Myc tags of SEQ ID NO: 49 can comprise the following sequence: EVKLQQSGGGLVQPGGSLTLSCVTSGFTFSTYGMDWVRQAPGKGFEWVASISFGGGITNY
GDFVKGRFTISRDNAKNTLYLHMNGLKPDDTAVYYCQMGSKRGQGTQVTVSSEPQTPKPQ
PQPQPAAAHHHHHHGAAEQKLISEEDLNGAA.

II.A. Antibody Variants

A targeting antibody of the presently disclosed subject matter comprises an antibody identified by the methods disclosed herein. In some embodiments, an antibody targeting ligand comprises a VHH domain. The presently disclosed subject matter also provides in some embodiments an isolated nucleic acid that encodes a VHH antibody fragment.

When phage-displayed antibodies bind to an antigen, they can be affinity-purified using the antigen. These affinity-purified phage can then be used to infect and introduce the antibody gene back into E. coli. The E. coli can then be grown and induced to express a soluble, non-phage-displayed, antigen-specific recombinant antibody.

The term “isolated”, as used in the context of a nucleic acid or polypeptide, indicates that the nucleic acid or polypeptide exists apart from its native environment and is not a product of nature. An isolated nucleic acid or polypeptide can exist in a purified form or can exist in a non-native environment such as a transgenic host cell.

The term “conservatively substituted variant” refers to an antibody comprising an amino acid residue sequence substantially identical to a sequence of a reference ligand of a target in which one or more residues have been conservatively substituted with a functionally similar residue and which displays the targeting activity as described herein. The phrase “conservatively substituted variant” also includes antibodies wherein a residue is replaced with a chemically derivatized residue, provided that the resulting peptide displays targeting activity as disclosed herein.

Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

Antibodies of the presently disclosed subject matter also include amino acid sequences comprising one or more additions and/or deletions or residues relative to the sequence of a VHH domain, such as those whose sequence is disclosed herein, so long as the requisite targeting activity of the peptide is maintained. The term “fragment” refers to an amino acid residue sequence shorter than that of a sequence of the presently disclosed subject matter, e.g. VHH domains, or of a wild-type or full-length sequence.

In some embodiments, the derivatives, fragments and variants of the VHH domains provided herein have the same or substantially the same immunogenic properties as the VHH domains from which they are derived. For example, a derivative, fragment or variant of a given VHH domain can have substantially the same binding activity to EGFR as the VHH domain. In some embodiments, derivatives, fragments or variants of a given VHH domain can be equally as useful, or have substantially equivalent utility to VHH domains, as targeting ligands for use in targeting cancer cells or tumors, or for use in therapeutic compositions, diagnostic compositions, and combinations thereof.

Fragments, variants or derivatives of the presently disclosed targeting ligands or VHH domains can be tested for their immunogenicity and/or binding activity using standard assays know to those of ordinary skill in the art. For example, competitive binding assays can be used to compare the immunogenicity of an antibody fragment with one or more disclosed VHH domains. A competitive binding assay can rely on the ability of a labeled standard antibody to compete with a test antibody fragment for binding with a limited amount of antigen. In some embodiments, sandwich-based assays can be used to determine the immunogenicity of an antibody fragment, variant or derivative. Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

Additional residues can also be added at either terminus for the purpose of providing a “linker” by which the VHH domains of the presently disclosed subject matter can be conveniently affixed to a label or solid matrix, or carrier. Amino acid residue linkers are usually at least one residue and can be 40 or more residues, more often 1 to 10 residues. Typical amino acid residues used for linking are tyrosine, cysteine, lysine, glutamic and aspartic acid, or the like. In addition, a peptide can be modified by terminal-NH₂ acylation (e.g., acetylation, or thioglycolic acid amidation) or by terminal-carboxylamidation (e.g., with ammonia, methylamine, and the like terminal modifications). Terminal modifications are useful, as is well known, to reduce susceptibility by proteinase digestion, and therefore serve to prolong the half life of the antibodies in solutions, particularly biological fluids where proteases can be present.

Nucleic Acids Encoding Targeting Antibodies. The terms “nucleic acid molecule” or “nucleic acid” each refer to deoxyribonucleotides or ribonucleotides and polymers thereof in single-stranded or double-stranded form. Unless specifically limited, the term encompasses nucleic acids containing known analogues of natural nucleotides that have similar properties as the reference natural nucleic acid. The terms “nucleic acid molecule” or “nucleic acid” can also be used in place of “gene”, “cDNA”, or “mRNA”. Nucleic acids can be synthesized, or can be derived from any biological source, including any organism.

The term “substantially identical”, as used herein to describe a degree of similarity between nucleotide sequences, refers to two or more sequences that have in some embodiments at least about 60%, in some embodiments at least about 65%, in some embodiments at least about 70%, in some embodiments at least about 75%, in some embodiments at least about 80%, in some embodiments at least about 85%, in some embodiments at least about 90%, in some embodiments at least about 93%, in some embodiments at least about 95%, in some embodiments at least about 96%, in some embodiments at least about 97%, in some embodiments at least about 98%, and in some embodiments at least about 99% nucleotide identity, as measured using one of the following sequence comparison algorithms (described hereinbelow) or by visual inspection. The substantial identity exists in nucleotide sequences of in some embodiments at least about 100 residues, in some embodiments at least about 150 residues, and in some embodiments in nucleotide sequences comprising a full length coding sequence.

Thus, substantially identical sequences can comprise mutagenized sequences, including sequences comprising silent mutations, or variably synthesized sequences. A mutation or variant sequence can comprise a single base change.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

An exemplary nucleotide sequence that can be employed for hybridization studies or assays includes probe sequences that are complementary to or mimic at least an about 14 to 40 nucleotide sequence of a nucleic acid molecule of the presently disclosed subject matter. For this purpose, a probe comprises a region of the nucleic acid molecule other than a sequence encoding a common immunoglobulin region. Thus, a probe comprises in some embodiments a sequence encoding a domain of the antibody that comprises an antigen-binding site. In some embodiments, probes comprise 14 to 20 nucleotides, or even longer where desired, such as 30, 40, 50, 60, 100, 200, 300 nucleotides or up to the full length of a region that encodes an antigen binding site. Such fragments can be readily prepared by, for example, chemical synthesis of the fragment, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes. Elsevier, N.Y. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook & Russell (2001) Molecular Cloning: a Laboratory Manual, 3rd ed. Cold Spring Harbor Laboratory Press, Cold Spring Harbor, N.Y., for a description of SSC buffer.

Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to identify nucleotide sequences that are substantially identical to reference nucleotide sequences of the presently disclosed subject matter: in some embodiments a probe nucleotide sequence hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 1×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 0.5×SSC, 0.1% SDS at 50° C.; in some embodiments a probe and target sequence hybridize in 7% sodium dodecyl sulfate (SDS), 0.5 M NaPO₄, 1 mM EDTA at 50° C. followed by washing in 0.1×SSC, 0.1% SDS at 50° C.; and in some embodiments a probe and target sequence hybridize in 7% sodium dodecyl sulfate (SDS), 0.5 M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further hereinbelow. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues. See Batzer et al. (1991) Nucleic Acids Res 19:5081; Ohtsuka et al. (1985) J Biol Chem 260:2605-2608; Rossolini et al. (1994) Mol Cell Probes 8:91-98994.

The term “subsequence” refers to a sequence of nucleic acids that comprises a part of a longer nucleic acid sequence. An exemplary subsequence is a probe, described hereinabove, or a primer. The term “primer” as used herein refers to a contiguous sequence comprising in some embodiments about 8 or more deoxyribonucleotides or ribonucleotides, in some embodiments about 10-20 nucleotides, and in some embodiments about 20-30 nucleotides of a selected nucleic acid molecule. The primers of the presently disclosed subject matter encompass oligonucleotides of sufficient length and appropriate sequence so as to provide initiation of polymerization on a nucleic acid molecule of the presently disclosed subject matter.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions are also known in the art. See e.g., Sambrook & Russell (2001) Molecular Cloning: a Laboratory Manual, 3rd ed. Cold Spring Harbor Laboratory Press, Cold Spring Harbor, N.Y.; Silhavy et al. (1984) Experiments with Gene Fusions. Cold Spring Harbor Laboratory, Cold Spring Harbor, N.Y.; Glover & Hames (1995) DNA Cloning: A Practical Approach, 2nd ed. IRL Press at Oxford University Press, Oxford/New York; Ausubel (1995) Short Protocols in Molecular Biology, 3rd ed. Wiley, New York.

TABLE 2
Functionally Equivalent Codons

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glumatic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | ACG | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It will also be understood by those of skill in the art that amino acid and nucleic acid sequences can include additional residues, such as additional N- or C-terminal amino acids or 5′ or 3′ nucleic acid sequences, and yet still be essentially as set forth in one of the sequences disclosed herein. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

Antibody Polypeptides. The term “substantially identical”, as used herein to describe a level of similarity between polypeptides comprising an antibody targeting ligand refers to a sequence having in some embodiments at least about 45%, in some embodiments at least about 50%, in some embodiments at least about 60%, in some embodiments at least about 70%, in some embodiments at least about 80%, in some embodiments at least about 90%, in some embodiments at least about 95%, in some embodiments at least about 96%, in some embodiments at least about 97%, in some embodiments at least about 98%, and in some embodiments at least about 99% sequence identity to a given sequence, when compared over the full length of the polypeptide. The term “full length”, as used herein to describe an antibody targeting ligand, comprises an amino acid sequence having a number of amino acids as set forth in a sequence in Table 1, for example. Methods for determining percent identity are defined herein.

Substantially identical polypeptides can also encompass two or more polypeptides sharing a conserved three-dimensional structure. Computational methods can be used to compare structural representations, and structural models can be generated and easily tuned to identify similarities around important active sites or ligand binding sites. See Saqi et al. (1999) Bioinformatics 15:521-522; Barton (1998) Acta Crystallogr D Biol Crystallogr 54:1139-1146; Henikoff et al. (2000) Electrophoresis 21:1700-1706; Huang et al. (2000) Pac Symp Biocomput 5:227-238.

Substantially identical proteins also include proteins comprising an amino acid sequence comprising amino acids that are functionally equivalent to amino acids of a given sequence. The term “functionally equivalent” in the context of amino acid sequences is known in the art and is based on the relative similarity of the amino acid side-chain substituents. Henikoff & Henikoff (1992) Proc Natl Acad Sci USA 89:10915-10919; Henikoff et al. (2000) Electrophoresis 21:1700-1706. Relevant factors for consideration include side-chain hydrophobicity, hydrophilicity, charge, and size. For example, arginine, lysine, and histidine are all positively charged residues; that alanine, glycine, and serine are all of similar size; and that phenylalanine, tryptophan, and tyrosine all have a generally similar shape. By this analysis, described further hereinbelow, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle (1982) J Mol Biol 157:105-132). It is known that certain amino acids can be substituted for other amino acids having a similar hydropathic index or score and still retain a similar biological activity, for example binding activity. In making changes based upon the hydropathic index, amino acids can be substituted whose hydropathic indices are in some embodiments within ±2 of the original value, in some embodiments within ±1 of the original value, and in some embodiments within ±0.5 of the original value.

It is also understood in the art that the substitution of like amino acids can be made effectively on the basis of hydrophilicity. U.S. Pat. No. 4,554,101 describes that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, amino acids can be substituted whose hydrophilicity values are in some embodiments within ±2 of the original value, in some embodiments within ±1 of the original value, and in some embodiments within ±0.5 of the original value.

The term “substantially identical” also encompasses polypeptides that are biologically functional equivalents. The term “functional”, as used herein to describe antibody-based targeting ligands, refers two or more antibodies that are immunoreactive with a same target molecule. In some embodiments, the two or more antibodies specifically bind a same target molecule and substantially lack binding to a control antigen.

The term “specifically binds”, when used to describe binding of an antibody to a target molecule, refers to binding to a target molecule in a heterogeneous mixture of other polypeptides.

The phases “substantially lack binding” or “substantially no binding”, as used herein to describe binding of an antibody to a control polypeptide or sample, refers to a level of binding that encompasses non-specific or background binding, but does not include specific binding.

Techniques for detecting antibody-target molecule complexes are known in the art and include but are not limited to centrifugation, affinity chromatography, ELISA, immunoprecipitation, flow cytometry and other immunochemical methods as known to those of ordinary skill in the art and as disclosed herein.

The presently disclosed subject matter also provides functional fragments of an antibody targeting polypeptide. Such functional portion need not comprise all or substantially all of the amino acid sequence of VHH domains disclosed herein.

The presently disclosed subject matter also includes functional polypeptide sequences that are longer sequences than that of a VHH domain disclosed herein. For example, one or more amino acids can be added to the N-terminus or C-terminus of an antibody targeting ligand. Methods of preparing such proteins are known in the art. In some embodiments, the VHH domains of the presently disclosed subject matter can be in the form of dimers and in some embodiments other multimeric formations. In some embodiments tumor accumulation of a small antibody is improved by increasing its molecular weight by dimerization. In addition to making homodimeric constructs, heterodimeric constructs comprising two different VHH domains can be constructed in some embodiments. In some embodiments, in order to confer conformational flexibility on the molecule, two domains can be connected by a linker, as discussed herein.

Isolated polypeptides and recombinantly produced polypeptides can be purified and characterized using a variety of standard techniques that are known to the skilled artisan. See e.g., Schroder & Lübke (1965) The Peptides, Academic Press, New York; Schneider & Eberle (1993) Peptides, 1992: Proceedings of the Twenty-Second European Peptide Symposium, Sep. 13-19, 1992, Interlaken, Switzerland, Escom, Leiden; Bodanszky (1993) Principles of Peptide Synthesis, 2nd rev. ed. Springer-Verlag, Berlin/New York; Ausubel (1995) Short Protocols in Molecular Biology, 3rd ed. Wiley, New York.

Nucleotide and Amino Acid Sequence Comparisons. The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence, or a given sequence as disclosed herein, by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, for example, by the local homology algorithm of Smith & Waterman (1981) Adv Appl Math 2:482-489, by the homology alignment algorithm of Needleman & Wunsch (1970) J Mol Biol 48:443-453, by the search for similarity method of Pearson & Lipman (1988) Proc Natl Acad Sci USA 85:2444-2448, by computerized implementations of these algorithms (e.g., programs available in the DISCOVERY STUDIO® package from Accelrys, Inc., San Diego, Calif., United States of America), or by visual inspection. See generally Ausubel (1995) Short Protocols in Molecular Biology, 3rd ed. Wiley, New York.

An exemplary algorithm for determining percent sequence identity and sequence similarity is the BLAST algorithm, which is described in Altschul et al. (1990) J Mol Biol 215:403-410. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul (1993) Proc Natl Acad Sci USA 90:5873-5877. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

II.B. Antibody Derivatives

Affinity maturation: To isolate higher affinity derivatives of first generation VHH domains, an affinity maturation method developed by Chowdhury and Pastan is used (Chowdhury et al. (1999) Nat. Biotechnol. 17:568-572; Yau et al. (2005) J. Immunol. Methods 297:213-224). The method mimics somatic hypermutation by random mutagenesis of hot spots in the DNA encoding the complementary-determining regions (CDRs) of an antibody. VHH domains contain three CDRs and although in one case, specificity and high affinity binding was shown to be conferred by CDR3 alone (Desmyter et al. (2001) J. Biol. Chem. 276:26285-26290), crystal structures and domain swapping experiments confirm the relevance of all 3 CDRs for antigen recognition and affinity (De Genst et al. (2006) Proc. Natl. Acad. Sci. U.S.A. 103:4586-4591; Saerens et al. (2005) J. Mol. Biol. 352:597-607). Two mutational hotspot motifs are the consensus sequences (A/G)-G-(C/T)-(A/T) and AG(C/T). Therefore, for affinity maturation of EGFR-specific VHH domains, DNA sequence corresponding to CDRs1-3 are searched for these motifs and degenerate primers are designed that randomize the codons that overlap them.

Efforts can include changing five codons at once for a given VHH domain cloned in the pHEN1 vector. A library containing all possible combinations of 20 amino acids at five positions could be expected to have a theoretical diversity of 3×10⁶ members, a library size that is readily achievable using the QuikChange™ Multi Site-Directed Mutagenesis kit (Stratagene 200514). To make the library, degenerate primers covering the randomized regions, 1 primer per CDR, encoding a total of five randomized amino acids were designed. NNK coding was used, where N represents equimolar ratios of A, C, G, or T, and K represents G or T. The NNK scheme uses 32 codons to encode 20 amino acids; the frequency of each amino acid is once (C, D, E, F, H, I, K, M, N, Q. W, Y), twice (A, G, P, V, T), or three times (L, R, S) per codon. The manufacturer's protocol for the QuickChange® kit (as described in Hogrefe et al. (2002) Biotechniques 33:1158-1165) was followed.

The library is introduced into E. coli TG1 by electroporation and the phage rescued by established methods (Barbas, C. F., 3rd, Burton, D. R., Scott, J. K., and Silverman, G. J. Phage Display: A Laboratory Manual. Cold Spring Harbor, N.Y.: Cold Spring Harbor Laboratory Press, 2001).

Selection and assessment of second generation VHH domains: In order to select higher affinity VHH-phage than the parent phage, 10 pmole of EGFR extracellular domain (ECD) is bound in 0.1 M NaHCO₃ pH 8.5 in a microplate well at 4° C. overnight. The well is blocked for 1 hr with 1% BSA in the same buffer. The well is washed with TBST and ˜10¹¹ library phage added. The phage is allowed to bind the target for 30 minutes at room temperature, after which they are removed. The plate is washed eight times with 250 μl PBST quickly to remove bulk phage, then incubated three times successively with 250 μl PBST for 1 hr each at room temperature. Phage remaining on the target are eluted by alternating acid and base treatment, the solution is neutralized and E. coli infected with the eluted phage for amplification and rescue. These rounds of selection are done, and then specificity and relative affinities of individual phage is assessed, in comparison with the parental VHH-phage, by ELISA on immobilized EGFR ECD. The phage with the highest apparent affinities are identified and used to infect E. coli HB2151 to permit the expression of free VHH domains. The VHH domains are purified by immobilized metal affinity chromatography. Affinity constants of the purified VHH domains are obtained by surface plasmon resonance using immobilized, purified EGFR ECD, and by Scatchard analysis on a cell line that expresses the EGFR, such as A431.

In some embodiments, the derivatives, fragments and variants of the VHH domains provided herein have the same or substantially the same immunogenic properties as the VHH domains from which they are derived. For example, a derivative, fragment or variant of a given VHH domain can have substantially the same binding activity to EGFR as the VHH domain. In some embodiments, derivatives, fragments or variants of a given VHH domain can be equally as useful, or have substantially equivalent utility to VHH domains, as targeting ligands for use in targeting cancer cells or tumors, or for use in therapeutic compositions, diagnostic compositions, and combinations thereof.

### III. Detection, Treatment, and Imaging

The presently disclosed subject matter provides in some embodiments methods and compositions for guided active agent delivery to a target cell or tissue (e.g. a cancer cell or a tumor) in a subject. The term “active agent” as used herein refers to any substance having biological or detectable activity. Thus, the term “active agent” includes a therapeutic agent, a diagnostic agent, or a combination thereof. The term “active agent” also includes any substance that is desirably delivered to a tumor.

In accordance with the presently disclosed subject matter, compositions can be used to deliver therapeutic agents to target tissues. A therapeutic composition of the presently disclosed subject matter can comprise one or more targeting ligands and a therapeutic agent, such that the therapeutic agent can be selectively targeted to a target tissue such as a tumor. Representative therapeutic agents include a radionuclide, a cytotoxin, and a chemotherapeutic agent. In some embodiments, a VHH domain of the presently disclosed subject matter can act as a therapeutic agent itself.

Also in accordance with the presently disclosed subject matter, a composition can further comprise a detectable label. In one embodiment, the detectable label is detectable in vivo. In this embodiment, the detectable label comprises a label that can be detected using magnetic resonance imaging, scintigraphic imaging, ultrasound, or fluorescence. An exemplary detectable label that can be used for detection is a radionuclide.

Thus, in some embodiments, a composition is prepared, the composition comprising a targeting ligand as disclosed herein and a diagnostic agent. In some embodiments, the composition can be used for the detection of a tumor in a subject by administering to the subject a targeting ligand of the presently disclosed subject matter, wherein the ligand comprises a detectable label; and detecting the detectable label, whereby a tumor is detected and visualized.

In some embodiments, a method for determining the presence of a tumor can comprise biopsying a suspected tumor; contacting a targeting ligand of the presently disclosed subject matter with the suspected tumor, wherein the ligand comprises a detectable label; in some embodiments rinsing to remove unbound ligand; and detecting the detectable label, whereby the detection of the targeting ligand on the biopsy of suspected tumor determines that the suspected tumor is a tumor. In some embodiments, the determination of the presence of a tumor is performed in vitro. In some embodiments the targeting ligand or composition is subjected to the biopsy of suspected tumor for a time sufficient for the targeting ligand to bind the suspected tumor. In some embodiments the targeting ligand binds EGFR on the suspected tumor, and particularly on the surface of tumor cells. In some embodiments the biopsy of suspected tumor is rinsed before detection to remove any unbound composition or targeting ligand. In some embodiments, determination of the presence of a tumor can further comprise characterizing the tumor.

In some embodiments, a therapeutic composition can additionally comprise a detectable label, in some embodiments a label that can be detected in vivo. The biodistribution of the therapeutic composition so prepared can be monitored following administration to a subject.

Compositions of the presently disclosed subject matter can be monovalent (e.g., they comprise an antibody that binds to only one epitope present on EGFR or other target) or polyvalent. As used herein, a “polyvalent composition” refers to a composition that comprises at least two different ligands (for example, antibodies) that bind to at least two different targets, for example EGFR and another target. Additionally, a “polyvalent composition” can refer to a composition that comprises at least two or more of the same ligand that can bind to more than one target molecule but at the same location within each target molecule.

Methods for preparation, labeling, and guided drug delivery using targeting ligands of the presently disclosed subject matter are described further herein. See, e.g., the Examples.

III.A. Therapeutic Compositions

In accordance with the methods of the presently disclosed subject matter, a therapeutic agent can also comprise a cytotoxic agent, a chemotherapeutic agent, a radionuclide, or any other anti-tumor molecule. Studies using ligand/drug conjugates have demonstrated that a chemotherapeutic agent can be linked to a ligand to produce a conjugate that maintains the binding specificity of the ligand and the therapeutic function of the agent. For example, doxorubicin has been linked to antibodies or peptides and the ligand/doxorubicin conjugates display cytotoxic activity (Shih et al. (1994) Cancer Immunol Immunother 38:92-98; Sivam et al. (1995) Cancer Res 55:2352-2356; Lau et al. (1995) Bioorg Med Chem 3:1299-1304, PCT International Publication No. WO 98/10795). Similarly, other anthracyclines, including idarubicin and daunorubocin, have been chemically conjugated to antibodies, which have facilitated delivery of effective doses of the agents to tumors (Aboud-Pirak et al. (1989) Biochem Pharmacol 38:641-648; Rowland et al. (1993) Cancer Immunol Immunother 37:195-202). Other chemotherapeutic agents include cis-platinum (Schechter et al. (1991) Intl J Cancer 48:167-172), methotrexate (Shawler et al. (1988) J Biol Response Mod 7:608-618) and mitomycin-C (Dillman et al. (1989) Mol Biother 1:250-255).

In some embodiments of the presently disclosed subject matter, a therapeutic agent comprises a radionuclide. Radionuclides can be effectively conjugated to antibodies (Hartmann et al. (1994) Cancer Res 54:4362-4370; Buchsbaum et al. (1995) Cancer Res 55:5881s-5887s), small molecule ligands (Wilbur (1992) Bioconjug Chem 3:433-470; Fjalling et al. (1996) J Nucl Med 37:1519-1521), and peptides (Boerman et al. (2000) Semin Nucl Med 30:195-208; Krenning & de Jong (2000) Ann Oncol 11:267-271; Kwekkeboom et al. (2000) J Nucl Med 41:1704-1713; Virgolini et al. (2001) Q J Nucl Med 45:153-159), such that administration of the conjugated radionuclide promotes tumor regression. Representative therapeutic radionuclides and methods for preparing a radionuclide-labeled agent are described further hereinbelow under the heading Scinitgraphic Imaging. For therapeutic methods of the presently disclosed subject matter, a representative radionuclide comprises ¹³¹I.

Additional anti-tumor agents that can be conjugated to the targeting ligands disclosed herein and used in accordance with the therapeutic methods of the presently disclosed subject matter include but are not limited to alkylating agents such as melphalan and chlorambucil (Aboud-Pirak et al. (1989) Biochem Pharmacol 38:641-648; Rowland et al. (1993) Cancer Immunol Immunother 37:195-202; Smyth et al. (1987) Immunol Cell Biol 65:315-321), vinca alkaloids such as vindesine and vinblastine (Aboud-Pirak et al. (1989) Biochem Pharmacol 38:641-648; Starling et al. (1992) Bioconjug Chem 3:315-322), antimetabolites such as 5-fluorouracil, 5-fluorouridine and derivatives thereof (Krauer et al. (1992) Cancer Res 52:132-137; Henn et al. (1993) J Med Chem 36:1570-1579).

III.B. Preparation of a Therapeutic and/or Diagnostic Composition

The presently disclosed subject matter also provides a method for preparing a composition for guided active agent delivery. In some embodiments, the method comprises conjugating the ligand to an active agent, whereby a composition for guided active agent delivery is prepared. An active agent can further comprise a carrier and can be formulated in any manner suitable for administration to a subject. In some embodiments, the method employs a targeting ligand comprising any one of the VHH sequences of Table 1.

Carriers. The compositions of the presently disclosed subject matter can further comprise a carrier to facilitate composition preparation and administration. Any suitable delivery vehicle or carrier can be used, including but not limited to a microcapsule, for example a microsphere or a nanosphere (Manome et al. (1994) Cancer Res 54:5408-5413; Saltzman & Fung (1997) Adv Drug Deliv Rev 26:209-230), a glycosaminoglycan (U.S. Pat. No. 6,106,866), a fatty acid (U.S. Pat. No. 5,994,392), a fatty emulsion (U.S. Pat. No. 5,651,991), a lipid or lipid derivative (U.S. Pat. No. 5,786,387), collagen (U.S. Pat. No. 5,922,356), a polysaccharide or derivative thereof (U.S. Pat. No. 5,688,931), a nanosuspension (U.S. Pat. No. 5,858,410), a polymeric micelle or conjugate (Goldman et al. (1997) Cancer Res 57:1447-1451 and U.S. Pat. Nos. 4,551,482, 5,714,166, 5,510,103, 5,490,840, and 5,855,900), and a polysome (U.S. Pat. No. 5,922,545).

Conjugation of Targeting Ligands. Antibody sequences can be coupled to active agents or carriers using methods known in the art, including but not limited to carbodiimide conjugation, esterification, sodium periodate oxidation followed by reductive alkylation, and glutaraldehyde crosslinking (Goldman et al. (1997) Cancer Res. 57:1447-1451; Cheng (1996) Hum. Gene Ther. 7:275-282; Neri et al. (1997) Nat. Biotechnol. 15:1271-1275; Nabel (1997) Vectors for Gene Therapy. In Current Protocols in Human Genetics, John Wiley & Sons, New York; Park et al. (1997) Adv. Pharmacol. 40:399-435; Pasqualini et al. (1997) Nat. Biotechnol. 15:542-546; Bauminger & Wilchek (1980) Meth. Enzymol. 70:151-159; U.S. Pat. No. 6,071,890; and European Patent No. 0 439 095).

Formulation. A therapeutic composition, a diagnostic composition, or a combination thereof, of the presently disclosed subject matter comprises in some embodiments a pharmaceutical composition that includes a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS in the range of in some embodiments 0.1 to 10 mg/ml, in some embodiments about 2.0 mg/ml; and/or mannitol or another sugar in the range of in some embodiments 10 to 100 mg/ml, in some embodiments about 30 mg/ml; and/or phosphate-buffered saline (PBS). Any other agents conventional in the art having regard to the type of formulation in question can be used. In some embodiments, the carrier is pharmaceutically acceptable. In some embodiments the carrier is pharmaceutically acceptable for use in humans.

III.C. Administration

Suitable methods for administration of a therapeutic composition, a diagnostic composition, or combinations thereof of the presently disclosed subject matter include but are not limited to intravascular, subcutaneous, or intratumoral administration. Further, upon a review of the instant disclosure, it is understood that any site and method for administration can be chosen, depending at least in part on the species of the subject to which the composition is to be administered. For delivery of compositions to pulmonary pathways, compositions can be administered as an aerosol or coarse spray.

For therapeutic applications, a therapeutically effective amount of a composition of the presently disclosed subject matter is administered to a subject. A “therapeutically effective amount” is an amount of the therapeutic composition sufficient to produce a measurable biological response (e.g., a cytotoxic response, or tumor regression). Actual dosage levels of active ingredients in a therapeutic composition of the presently disclosed subject matter can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. In some embodiments of the presently disclosed subject matter, a minimal dose is administered, and dose is escalated in the absence of dose-limiting toxicity. Determination and adjustment of a therapeutically effective dose, as well as evaluation of when and how to make such adjustments, are known to those of ordinary skill in the art.

For diagnostic applications, a detectable amount of a composition of the presently disclosed subject matter is administered to a subject. A “detectable amount”, as used herein to refer to a diagnostic composition, refers to a dose of such a composition that the presence of the composition can be determined in vivo or in vitro. A detectable amount will vary according to a variety of factors, including but not limited to chemical features of the agent being labeled, the detectable label, labeling methods, the method of imaging and parameters related thereto, metabolism of the labeled agent in the subject, the stability of the label (e.g. the half-life of a radionuclide label), the time elapsed following administration of an active agent and/or labeled antibody prior to imaging, the route of drug administration, the physical condition and prior medical history of the subject, and the size and longevity of the tumor or suspected tumor. Thus, a detectable amount can vary and can be tailored to a particular application. After study of the present disclosure, including the Appendix, it is within the skill of one in the art to determine such a detectable amount.

III.D. Monitoring Distribution In Vivo

In some embodiments of the presently disclosed subject matter, a diagnostic and/or therapeutic composition for guided delivery comprises a label that can be detected in vivo. The term “in vivo”, as used herein to describe imaging or detection methods, can refer to generally non-invasive methods such as scintigraphic methods, magnetic resonance imaging, ultrasound, or fluorescence, each described briefly hereinbelow. The term “non-invasive methods” does not exclude methods employing administration of a contrast agent to facilitate in vivo imaging.

The label can be conjugated or otherwise associated with a targeting ligand (e.g., any one of the VHH domains disclosed herein), a therapeutic, a diagnostic agent, a carrier, or combinations thereof. Following administration of the labeled composition to a subject, and after a time sufficient for binding, the biodistribution of the composition can be visualized. The term “time sufficient for binding” refers to a temporal duration that permits binding of the labeled agent to a target molecule.

In some embodiments the presently disclosed subject matter provides methods for imaging a target tissue in a subject. In some embodiments one or more targeting ligands of the presently disclosed subject matter can be administered to a subject, wherein the targeting ligands further comprise an in vivo detectable label. Detection of the targeting ligand with an in vivo detectable label can provide for the detection, imaging, identification and/or diagnosis of a target tissue or tumor.

Scintigraphic Imaging. Scintigraphic imaging methods include SPECT (Single Photon Emission Computed Tomography), PET (Positron Emission Tomography), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems comprise an array of detectors in a ring that also detect radioactivity in multiple dimensions. PET-CT is an instrument that can carry out PET and CT (Computed Tomography) simultaneously.

Other imaging instruments suitable for practicing the method of the presently disclosed subject matter, and instruction for using the same, are readily available from commercial sources. Both PET and SPECT systems are offered by ADAC of Milpitas, Calif., United States of America, and Siemens of Hoffman Estates, Ill., United States of America. Related devices for scintigraphic imaging can also be used, such as a radio-imaging device that includes a plurality of sensors with collimating structures having a common source focus.

When scintigraphic imaging is employed, the detectable label comprises in some embodiments a radionuclide label, in some embodiments a radionuclide label selected from the group including but not limited to ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹ mtellurium, ^{122m}tellurium, ^{125m}tellurium, ¹⁶⁵thulium, ¹⁶⁷thulium, ¹⁶⁸thulium, and nitride or oxide forms derived there from. In some embodiments the radionuclide label comprises ¹³¹iodine or ^{99m}technetium.

Methods for radionuclide labeling of a molecule so as to be used in accordance with the disclosed methods are known in the art. For example, a targeting molecule can be derivatized so that a radioisotope can be bound directly to it (Yoo et al. (1997) J Nucl Med 38:294-300). Alternatively, a linker can be added to enable conjugation. Representative linkers include diethylenetriamine pentaacetate (DTPA)-isothiocyanate, succinimidyl 6-hydrazinium nicotinate hydrochloride (SHNH), and hexamethylpropylene amine oxime (HMPAO) (Chattopadhyay et al. (2001) Nucl. Med. Biol. 28:741-744; Sagiuchi et al. (2001) Ann. Nucl. Med. 15:267-270; Dewanjee et al. (1994) J. Nucl. Med. 35:1054-1063; U.S. Pat. No. 6,024,938). Additional methods can be found in U.S. Pat. No. 6,080,384; Hnatowich et al. (1996) J. Pharmacol. Exp. Ther. 276:326-334; and Tavitian et al. (1998) Nat. Med. 4:467-471.

When the labeling moiety is a radionuclide, stabilizers to prevent or minimize radiolytic damage, such as ascorbic acid, gentisic acid, or other appropriate antioxidants, can be added to the composition comprising the labeled targeting molecule.

Magnetic Resonance Imaging (MRI). Magnetic resonance image-based techniques create images based on the relative relaxation rates of water protons in unique chemical environments. As used herein, the term “magnetic resonance imaging” refers to magnetic source techniques including conventional magnetic resonance imaging, magnetization transfer imaging (MTI), proton magnetic resonance spectroscopy (MRS), diffusion-weighted imaging (DWI) and functional MR imaging (fMRI). See Rovaris et al. (2001) J Neurol Sci 186 Suppl 1:S3-9; Pomper & Port (2000) Magn Reson Imaging Clin N Am 8:691-713.

Contrast agents for magnetic source imaging include but are not limited to paramagnetic or superparamagnetic ions, iron oxide particles (Weissleder et al. (1992) Magn Reson Q 8:55-63; Shen et al. (1993) Magn Reson Med 29:599-604), and water-soluble contrast agents. Paramagnetic and superparamagnetic ions can be selected from the group of metals including iron, copper, manganese, chromium, erbium, europium, dysprosium, holmium and gadolinium. Representative metals are iron, manganese and gadolinium.

Those skilled in the art of diagnostic labeling recognize that metal ions can be bound by chelating moieties, which in turn can be conjugated to a therapeutic agent in accordance with the methods of the presently disclosed subject matter. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Alternatively, a contrast agent can be carried in a liposome (Schwendener (1992) Chimia 46:69-77).

Images derived used a magnetic source can be acquired using, for example, a superconducting quantum interference device magnetometer (SQUID, available with instruction from Quantum Design of San Diego, Calif., United States of America). See U.S. Pat. No. 5,738,837.

Ultrasound. Ultrasound imaging can be used to obtain quantitative and structural information of a target tissue, including a tumor. Administration of a contrast agent, such as gas microbubbles, can enhance visualization of the target tissue during an ultrasound examination. Preferably, the contrast agent can be selectively targeted to the target tissue of interest, for example by using an antibody fragment for guided delivery as disclosed herein. Representative agents for providing microbubbles in vivo include but are not limited to gas-filled lipophilic or lipid-based bubbles (e.g., U.S. Pat. Nos. 6,245,318, 6,231,834, 6,221,018, and 5,088,499). In addition, gas or liquid can be entrapped in porous inorganic particles that facilitate microbubble release upon delivery to a subject (U.S. Pat. Nos. 6,254,852 and 5,147,631).

Gases, liquids, and combinations thereof suitable for use with the presently disclosed subject matter include air; nitrogen; oxygen; carbon dioxide; hydrogen; nitrous oxide; an inert gas such as helium, argon, xenon or krypton; a sulphur fluoride such as sulphur hexafluoride, disulphur decafluoride or trifluoromethylsulphur pentafluoride; selenium hexafluoride; an optionally halogenated silane such as tetramethylsilane; a low molecular weight hydrocarbon (e.g. containing up to 7 carbon atoms), for example an alkane such as methane, ethane, a propane, a butane or a pentane, a cycloalkane such as cyclobutane or cyclopentane, an alkene such as propene or a butene, or an alkyne such as acetylene; an ether; a ketone; an ester; a halogenated low molecular weight hydrocarbon (e.g. containing up to 7 carbon atoms); or a mixture of any of the foregoing. Halogenated hydrocarbon gases can show extended longevity, and thus are preferred for some applications. Representative gases of this group include decafluorobutane, octafluorocyclobutane, decafluoroisobutane, octafluoropropane, octafluorocyclopropane, dodecafluoropentane, decafluorocyclopentane, decafluoroisopentane, perfluoropexane, perfluorocyclohexane, perfluoroisohexane, sulfur hexafluoride, and perfluorooctanes, perfluorononanes; perfluorodecanes, optionally brominated.

Attachment of targeting ligands to lipophilic bubbles can be accomplished via chemical crosslinking agents in accordance with standard protein-polymer or protein-lipid attachment methods (e.g., via carbodiimide (EDC) or thiopropionate (SPDP)). To improve targeting efficiency, large gas-filled bubbles can be coupled to a targeting ligand using a flexible spacer arm, such as a branched or linear synthetic polymer (U.S. Pat. No. 6,245,318). A targeting ligand can be attached to the porous inorganic particles by coating, adsorbing, layering, or reacting the outside surface of the particle with the targeting ligand (U.S. Pat. No. 6,254,852).

A description of ultrasound equipment and technical methods for acquiring an ultrasound dataset can be found in Coatney (2001) Ilar J 42:233-247; Lees (2001) Semin Ultrasound CT MR 22:85-105; and references cited therein.

Fluorescent Imaging. Non-invasive imaging methods can also comprise detection of a fluorescent label. An active agent comprising a lipophilic component (therapeutic agent, diagnostic agent, vector, or drug carrier) can be labeled with any one of a variety of lipophilic dyes that are suitable for in vivo imaging. See e.g. Fraser (1996) Meth Cell Biol 51:147-160; Ragnarson et al. (1992) Histochemistry 97:329-333; and Heredia et al. (1991) J Neurosci Meth 36:17-25. Representative labels include but are not limited to carbocyanine and aminostyryl dyes, preferably long chain dialkyl carbocyanines (e.g., Dil, DiO, and DID available from Molecular Probes Inc. of Eugene, Oreg., United States of America) and dialkylaminostyryl dyes.

Lipophilic fluorescent labels can be incorporated using methods known to one of skill in the art. For example VYBRANT™ cell labeling solutions are effective for labeling of cultured cells or other lipophilic components (Molecular Probes Inc. of Eugene, Oreg., United States of America).

A fluorescent label can also comprise sulfonated cyanine dyes, including Cy5.5 and Cy5 (available from Amersham of Arlington Heights, Ill., United States of America), IRD41 and IRD700 (available from Li-Cor, Inc. of Lincoln, Nebr.), NIR-1 (available from Dejindo of Kumamoto, Japan), and LaJolla Blue (available from Diatron of Miami, Fla., United States of America). See also Licha et al. (2000) Photochem Photobiol 72:392-398; Weissleder et al. (1999) Nat Biotechnol 17:375-378; and Vinogradov et al. (1996) Biophys J 70:1609-1617.

In addition, a fluorescent label can comprise an organic chelate derived from lanthanide ions, for example fluorescent chelates of terbium and europium (U.S. Pat. No. 5,928,627). Such labels can be conjugated or covalently linked to an active agent as disclosed therein.

For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

III.E. In Vitro Detection

The presently disclosed subject matter further provides methods for determining the presence of a tumor. In some embodiments the determination of the presence of a tumor can further coincide with diagnosing and/or characterizing a tumor. In some embodiments, a targeting ligand of the presently disclosed subject matter comprises a detectable label such as a fluorescent, epitope, or radioactive label, each described briefly hereinbelow. In some embodiments, determining the presence of a tumor comprises contacting the biopsy of suspected tumor with a composition comprising a targeting ligand of the presently disclosed subject matter, and further comprising a detectable label; in some embodiments rinsing the biopsy of suspected tumor to remove unbound composition; and detecting the composition bound to the biopsy of suspected tumor, whereby the detection of the composition on the biopsy of suspected tumor determines that the suspected tumor is a tumor. In some embodiments detection of the composition comprises detection of the targeting ligand comprising a detectable label using autoradiography or fluorescence.

Fluorescence. Any detectable fluorescent dye can be used, including but not limited to FITC (fluorescein isothiocyanate), FLUOR X™, ALEXA FLUOR®, OREGON GREEN®, TMR (tetramethylrhodamine), ROX α-rhodamine), TEXAS RED®, BODIPY® 630/650, and Cy5 (available from Amersham Pharmacia Biotech of Piscataway, N.J., United States of America, or from Molecular Probes Inc. of Eugene, Oreg., United States of America).

A fluorescent label can be detected directly using emission and absorbance spectra that are appropriate for the particular label used. Common research equipment has been developed for in vitro detection of fluorescence, including instruments available from GSI Lumonics (Watertown, Mass., United States of America) and Genetic MicroSystems Inc. (Woburn, Mass., United States of America). Most of the commercial systems use some form of scanning technology with photomultiplier tube detection. Criteria for consideration when analyzing fluorescent samples are summarized by Alexay et al. (1996) The PCT International Society of Optical Engineering 2705/63.

Detection of an Epitope. If an epitope label has been used, a protein or compound that binds the epitope can be used to detect the epitope. A representative epitope label is biotin, which can be detected by binding of an avidin-conjugated fluorophore, for example avidin-FITC. Alternatively, the label can be detected by binding of an avidin-horseradish peroxidase (HRP) streptavidin conjugate, followed by colorimetric detection of an HRP enzymatic product. The production of a colorimetric or luminescent product/conjugate is measurable using a spectrophotometer or luminometer, respectively.

Autoradioqraphic Detection. In the case of a radioactive label (e.g., ¹³¹I or ^{99m}Tc) detection can be accomplished by conventional autoradiography or by using a phosphorimager as is known to one of skill in the art. A representative autoradiographic method employs photostimulable luminescence imaging plates (Fuji Medical Systems of Stamford, Conn., United States of America). Briefly, photostimulable luminescence is the quantity of light emitted from irradiated phosphorous plates following stimulation with a laser during scanning. The luminescent response of the plates is linearly proportional to the activity.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1

### Materials and Methods

Epidermal growth factor receptor (EGFR) is overexpressed or mutated in a high percentage of tumors. The presently disclosed subject matter provides ligands targeted to EGFR for use in cancer diagnostic and therapeutic applications. The presently disclosed subject matter addresses the limitations and poor efficacy of monoclonal antibodies and other large antibody constructs that diffuse into tumors slowly. In order to develop lower molecular weight probes for EGFR and other tumor cell receptors, a llama was immunized with extracellular domains (ECDs) of EGFR and an oncogenic mutant receptor, EGFRvIII, and with extracts of tumor cell lines. A heavy chain variable domain (VHH domain)-phage library was constructed from the immune repertoire of the llama. At ˜16 kDa, the VHH domain is a tenth of the size of a monoclonal antibody and is the smallest antibody fragment that retains specificity. By affinity selection from this library, many VHH domains with specificity for EGFR were isolated. The VHH domains bind to whole cells expressing the receptor but not to control cells lacking the receptor and can immunoprecipitate EGFR from cell lysates. Some VHH domains have cross-specificity with existing anti-EGFR monoclonal antibodies and have reasonably high (nM) affinities. The llama-VHH domain library is also potentially a rich source of targeting agents directed toward other tumor cell receptors.

EGFR is a well-studied representative of this type of cancer marker. EGFR (or ErbB-1) is a member of a family of transmembrane tyrosine kinase receptors whose other members are HER2/c-neu (ErbB-2), Her3 (ErbB-3) and Her4 (ErbB-4). EGFR is a 170 kDa protein with an extracellular ligand binding domain, a membrane-spanning region and an intracellular tyrosine kinase domain (Ullrich et al. (1984) Nature 309:418-425). Upon ligand binding, the receptor forms a homodimer or heterodimer with another member of the receptor family (Schlessinger (2002) Cell 110:669-672). Ligand binding leads to activation of receptor tyrosine kinase activity, which triggers downstream growth-promoting signaling pathways (Fischer et al. (2003) Biochem Soc Trans 31:1203-1208). EGFR is then down-regulated by endocytic internalization, compartmentalization and degradation (Dikic (2003) Biochem Soc Trans 31:1178-1181).

Although EGFR is present on the surface of most normal cells, over-expression of wild type EGFR and expression of mutated EGFR have been associated with tumors of the lung, brain, breast, ovary, prostate and other cancers (Lynch et al. (2004) N Engl J Med 350:2129-2139; Hirsch et al. (2003) Lung Cancer 41:(Suppl 1) S29-42; Kuan et al. (2000) Brain Tumor Pathol 17:71-78; Kim & Muller (1999) Exp Cell Res 253:78-87; Maihle et al. (2002) Cancer Treat Res 107:247-258; Lorenzo et al. (2003) Clin Prostate Cancer 2:50-57). EGFR amplification has been found to be strongly correlated with tumor progression (Meert et al. (2003) Eur Respir J 21:611-615; Piyathilake et al. (2002) Clin Cancer Res 8:734-744; Selvaggi et al. (2004) Ann Oncol 15:28-32). Activating mutations of EGFR can be found in both the ECD and intracellular kinase domain. The EGFRvIII mutation is present in a significant fraction of non-small cell lung cancers, breast carcinomas and glioblastoma (Garcia de Palazzo et al. (1993) Cancer Research 53:3217-3220; Okamoto et al. (2003) Cancer Sci 94:50-56; Wikstrand et al. (1995) Cancer Research 55:3140-3148). EGFRvIII is characterized by a 267 amino acid deletion that creates a unique epitope in the ECD (Pedersen et al. (2001) Ann Oncol 12:745-760). This mutation renders the receptor ligand-independent, constitutively active and oncogenic. Other oncogenic mutations that occur in the intracellular domain have altered signaling properties (Padron et al. (2007) Cancer Res 67:7695-7702).

Monoclonal antibodies (mAbs) with specificity for EGFR are not optimal therapeutic or imaging reagents due to the slow tumor penetration of a 160 kDa protein. This is partly due to high interstitial pressure inside tumors which prevents convection of the antibody from blood vessels, and partly due to the slow intratumoral diffusion rates of a 160 kDa IgG molecule (Jain, R. K. Vascular and interstitial physiology of tumours: role in cancer detection and treatment. In: R. Bicknell, C. E. Lewis, and N. Ferrara (eds.), Tumour Angiogenesis, pp. 45-59. Oxford: Oxford University Press, 1997). It has been estimated that a molecule the size of IgG would require 1 week to reach an intratumoral concentration equal to one-half its concentration in the blood at a distance of 1 mm from the vascular endothelium, and months to penetrate completely a 1 cm tumor (˜10⁹ cells) (Teicher, B. A. (ed.) Physiological resistance to the treatment of solid tumors. New York: Marcel Dekker, Inc., 1993). In addition, some mAbs can be immunogenic (Hwang et al. (2005) Methods 36:3-10).

Accordingly, the presently disclosed subject matter provides in some embodiments a smaller, non-immunogenic imaging agent with fast tumor infiltration kinetics that retains the high specificity and affinity of an antibody. Stability to serum proteases and the ability to withstand renal clearance for several hours are further desirable properties of the disclosed targeting ligands.

The naturally occurring heavy chain antibodies of camels and llamas might, provide a source of low molecular weight yet specific tumor targeting agents (Hamers-Casterman et al. (1993) Nature 363:446-448). These antibodies are devoid of light chains; the antigen binding domain is comprised solely of the variable regions of two heavy chains. A 16 kDa monomeric protein derived from a single heavy chain variable region, termed the VHH domain, is the smallest antigen binding fragment known (Sheriff & Constantine (1996) Nat Struct Biol 3:733-736). These molecules are highly soluble when expressed in a recombinant system, do not aggregate as do some scFvs and are non-immunogenic in mice (Dumoulin et al. (2002) Protein Sci 11:500-515; Cortez-Retamozo et al. (2002) Int J Cancer 98:456-462. A VHH domain has been shown to cross the blood-brain barrier (Muruganandam et al. (2002) FASEB J 16:240-242).

The present co-inventors have constructed a llama-VHH domain library and show herein that large numbers of EGFR-specific VHH domains can be isolated from it with ease. This llama-VHH domain library is also potentially a source of targeting agents directed toward other tumor cell receptors. The current study is a detailed account of the isolation of EGFR-specific VHH domains and their biochemical characterization.

Materials

The EGFR mAb, H11, was purchased from Dako (Carpinteria, Calif., United States of America). All other EGFR-specific mAbs were a gift of Dr. D. Bigner, Duke University Medical Center, Durham, N.C., United States of America. Zinc Option medium, RPMI 1640 medium and Fetal Bovine Serum (FBS) were purchased from Gibco-Invitrogen (Invitrogen Corp., Carlsbad, Calif., United States of America). 96-well coated microplates were from BD Biosciences, San Jose, Calif., United States of America.

Cell Lines

The NR6M, NR6W and NR6V cell lines are Swiss mouse 3T3 cells transfected with human EGFRvIII, human EGFR wild type, or vector, respectively (Batra et al. (1995) Cell Growth and Differentiation 6:1251-1259). These three cell lines and the human glioblastoma cell line U87MG (Ponten & Macintyre (1968) Acta Pathol Microbiol Scand 74:465-486) were obtained from Dr. D. Bigner. All were grown in Zinc Option (Invitrogen Corp.)+10% FBS (Invitrogen Corp.). The human adenocarcinoma cell line ADLC-5M2 (Bepler et al. (1988) Differentiation 37:158-171) was obtained from Dr. G. Bepler from H. Lee Moffitt Cancer Center and Research Institute, Tampa, Fla., United States of America, and grown in RPMI 1640+10% FBS (Invitrogen Corp.). All cell lines were maintained at 37° C. in a 5% CO₂ incubator.

Llama Immunization

Llama immunization and subsequent care were carried out under an animal protocol approved by Duke University IACUC. ADLC-5M2 and U87MG cells were harvested with 5 mM EDTA in phosphate buffered saline (PBS) and proteins were extracted by incubation for 30 min at 4° C. with Mammalian Protein Extraction Reagent (M-PER; Pierce, Rockford, Ill., United States of America) containing protease inhibitors (Complete Protease Inhibitor Cocktail, Roche Applied Science, Indianapolis, Ind., United States of America). Cellular debris was removed by centrifugation at 16,000×g for 10 min at 4° C. The resultant lysate was stored at −80° C. until use. For immunization, purified recombinant proteins (100 μg each EGFR-ECD, and EGFRvIII ECD) and cell lysate proteins (50 μg U87MG and 80 μg ADLC-5M2) were added to adjuvant (Cedi Diagnostics, Lelystad, The Netherlands) at a ratio of 4 parts protein solution to 5 parts adjuvant, and mixed thoroughly to form an emulsion. The emulsion (total volume 2 ml) was then injected into a llama, one-half subcutaneously and one-half intramuscularly. The llama was boosted using the same protocol 25 days later, and blood was collected 39 days after the boost. Assay of the sera before and after the injections indicated a specific humoral response to EGFR.

### Example 2

### Library Construction

Peripheral blood lymphocytes were isolated using the Lymphoprep™ kit (Axis-Shield, Oslo, Norway). RNA was purified from 4.5×10⁷ lymphocytes using the versaGene™ Total RNA Purification kit (Gentra Systems, Inc., Minneapolis, Minn., United States of America), and 5 μg RNA was converted to cDNA using Transcriptor Reverse Transcriptase (Roche Applied Science). The variable domain of the heavy chain-only llama IgG was amplified by polymerase chain reaction (PCR). The amplification strategy was based on that described previously by Dekker et al. (2003) J Virol 77:12132-12139; van der Linden et al. (2000) J Immunol Methods 240:185-195). Two separate amplifications were performed using a common reverse primer (VhbackSfiI) and one of two different forward primers, one priming on the “short hinge region” (SHNotI) and one priming on the “long hinge region” (LHNotI) of this class of antibodies. The sequences of the primers were: VhbackSfiI: 5′-CATGCCATGACTCGCGGCCCAGCCGGCCATGGCCSAGGTSMARCTG CAGSAGTCWGG-3′ (SEQ ID NO: 51); SHNotI: 5′-TTTTCCTTTTGCGGCCGCGGCCGCGGAGCTGGGGTCTTCGCTGTGG TGCG-3′ (SEQ ID NO: 52); LHNotI: 5′-GGATTGGGTTGCGGCCGCTGGTTGTGGTTGTGGTTGTGGTTTTGGT GTCTGGGGTTC-3′ (SEQ ID NO: 53).

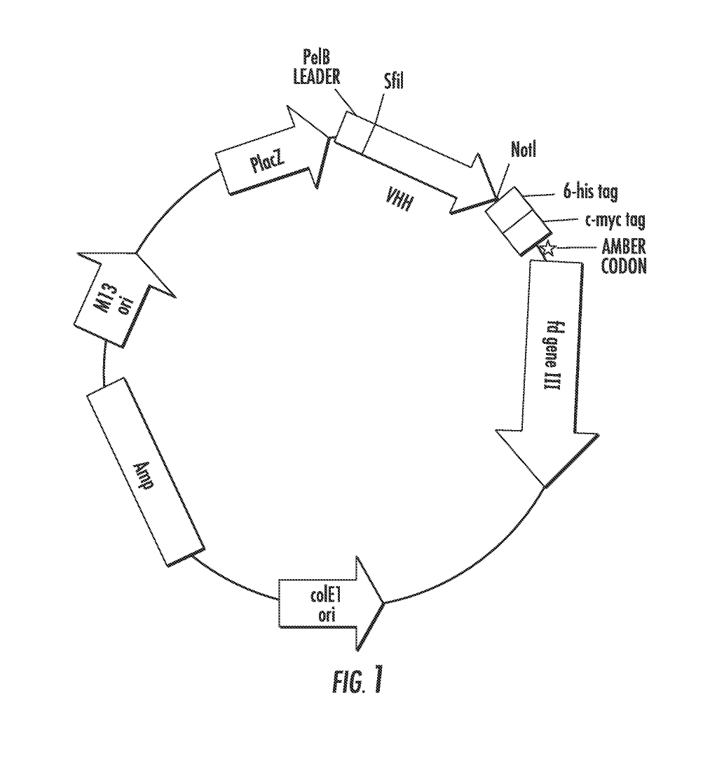

The PCR products were agarose gel purified and combined, digested with SfiI and NotI and ligated upstream of the bacteriophage fd gene III in SfiI/NotI cut phagemid vector pHEN1 (Hoogenboom et al. (1991) Nucleic Acids Res 19:4133-4137) modified to contain a carboxyl terminal 6-His tag (FIG. 1). This phagemid (pHEN1-H6) replicates as a plasmid in E. coli; when the library is subsequently “rescued” in the presence of “helper” phage, the VHH-gene III protein fusion is incorporated into the progeny phage as part of the tail assembly. The ligation mixture (2 μg total DNA) was introduced 25 ng at a time into electrocompetent E. coli TG1 cells (Stratagene, La Jolla, Calif., United States of America) using a MicroPulser™ electroporation apparatus (Bio-Rad, Hercules, Calif., United States of America). A total of approximately 1×10⁷ transformants were recovered, and the library was amplified and rescued with helper phage VCSM13 using established methods (Barbas, C. F., 3rd, Burton, D. R., Scott, J. K. & Silverman, G. S. Phage Display: A Laboratory Manual; Cold Spring Harbor Laboratory Press, Cold Spring Harbor, N.Y.; 2001).

### Example 3

### Purification of EGFR ECDs

High Five insect cells (Invitrogen Corp.) were infected with a viral stock containing either a EGFRvIII ECD or wild type EGFR ECD expression vector. After harvesting infected cells, culture supernatant was dialyzed against PBS, pH 7.4, passed through a 0.2 μm filter, and applied to an immunoaffinity column (Sepharose 4B conjugated to EGFR-specific mAb H11). The column was eluted with glycine HCl buffer, pH 3.0, and all fractions containing protein were combined and dialyzed against PBS.

### Example 4

### Affinity Selection

For each round of selection, 10 pmole of EGFR ECD (wild type or vIII mutant) in 0.1 M NaHCO₃ pH 8.5 was bound to a well of a “high binding” microplate (Greiner Bio-One North America, Inc., Monroe, N.C., United States of America) at 4° C. overnight. The wells were blocked for 1 hr with 1% BSA in the same buffer. The plate was washed with PBS plus 0.1% (v/v) TWEEN®-20 (PBST) and 1×10¹¹ library phage were added to the wells. Phage were allowed to bind to the target for 3 hr at room temperature, after which they were removed and the wells were washed 8 times with PBST. Phage were eluted from the target by alternating acid (50 mM glycine, pH 2.2) and base (100 mM triethylamine, pH 10) treatment, the solution was neutralized with Tris-HCl pH 7.4 and E. coli TG1 was infected with the eluted phage for amplification. Following phagemid amplification, phage rescue was performed to generate the input phage for the next round of selection. Four rounds of selection were performed.

### Example 5

### Cell ELISA NR6W, NR6M and NR6V were plated at an initial density of 5×10⁴ cells per well in 96-well poly-L-lysine, poly-D-lysine or collagen-coated microplates in serum-containing media. The microplates were incubated for 16 hours to allow surface attachment and all cell lines became confluent in this time. Media were removed, and cells were washed with PBS and fixed by adding 100 μl 0.025% glutaraldehyde (Sigma-Aldrich, Inc., St. Louis, Mo., United States of America) to each well for 2 min. The glutaraldehyde was removed and the cells were washed with PBS. For the ELISA, the plates were washed 4 times with PBST then 100 μl phage supernatant was added to each well. The plates were incubated at room temperature for 1 hour and washed 4 times with PBST. Anti-M13-HRP antibody conjugate (GE Healthcare, Piscataway, N.J., United States of America) was diluted 1:1000 in PBST and 100 μl was added to each well. After 1 hour incubation, the plates were washed and developed with 1 mM 2, 2′-azino-bis(3-ethylbenzthiazoline-6-sulfonic acid) in 50 mM citrate plus 0.03% (v/v) H₂O₂. The plates were read at 405 nm on a 96-well microplate reader (Tecan US, Inc, Durham, N.C., United States of America). For competition ELISA, antibodies were diluted in a microplate then transferred to the cell ELISA plate in a volume of 25 μl. Phage (75 μl) were immediately added, the plates were incubated for 1 hour, and were processed as described above.

### Example 6

### Immunoprecipitation

Free VHH domains were expressed in and purified from E. coli HB2151 periplasm by nickel affinity chromatography using His SpinTrap™ columns (GE Healthcare) according to the manufacturer's instructions. In HB2151, VHH domains are expressed as free soluble fragments due to lack of amber suppression at the VHH-gene IIIp junction. The purified VHH domains were used to pull down target protein from mammalian cell lysates by two different methods.

For plate pulldown, 20 μg of each of six EGFR-specific VHH domains and one random VHH clone were placed in individual wells of a nickel-chelate microplate (HIS-Select® HC; nominal capacity 4 μg; Sigma-Aldrich, Inc.); an eighth well received only buffer. The VHH domains were allowed to bind to the plate wells for 16 hr at 4° C., and then the wells were washed 3 times with Tris-buffered saline containing 0.1% (v/v) TWEEN®-20 (TBST). NR6W lysate (450 μg) made with M-PER reagent (Pierce) plus EDTA-free protease inhibitors (Roche Applied Science) was added to each well and allowed to incubate for 16 hr at 4° C. The lysate was removed, and the wells were washed 15 times with TBST. Protein remaining on the well surface was eluted in SDS-PAGE sample buffer containing 1% (v/v) β-mercaptoethanol and 100 mM imidazole, preheated to 95° C. Samples were subjected to SDS-PAGE and proteins were transferred to a PVDF membrane that was then probed with the anti-EGFR mAb H11 and a secondary goat-anti-mouse IgG-HRP conjugate. The blot was developed with SuperSignal West™ substrate (Pierce) and exposed to film.

For bead pulldown, VHH122 and negative control VHH RP2 were each coupled to CNBr-activated Sepharose 4B beads (GE Healthcare) according to the manufacturer's instructions and incubated with NR6W lysate for 3 hr at 4° C. The beads were washed 5 times with PBS and protein eluted with SDS-Protein Sample Buffer at 95° C. Eluted protein was subjected to SDS-PAGE and stained with Coomassie Blue; a single band unique to the VHH122 lane was excised from the gel. This protein was subjected to tryptic digestion and sequence analysis by mass spectrometry at the UNC/Duke Proteomics Center, Chapel Hill, N.C., United States of America.

### Example 7

### Surface Plasmon Resonance

The EGFR ECDs were coupled to BIACORE® CM5 chips and VHH domains were flowed over each protein in HEPES-buffered saline solution in the BIACORE® 3000 (GE Healthcare) at room temperature. Surface plasmon resonance measurements were taken and the binding and dissociation phases were fit by the instrument's software to obtain rate and affinity constants.

### Example 8

### Flow Cytometry

NR6W and NR6V cells were grown to confluence and cells were harvested with 0.02% (w/v) EDTA in PBS. The cells were washed with PBS and resuspended in Zinc Option medium (Invitrogen Corp.), with no serum. Cells (8×10⁵) were mixed with either VHH122 or the random control RP2 at 15 μg/ml and incubated for 1 hr on ice. The cells were washed two times each with 3 ml PBS, and then resuspended in a solution of FITC-conjugated mouse anti-His tag mAb (Abcam ab53178; Abcam, Inc., Cambridge, Mass., United States of America) diluted 1:100 in Zinc Option medium. After incubation for 30 min on ice, the cells were washed and analyzed in a BD FACScalibur® flow cytometer (BD Biosciences, San Jose, Calif., United States of America).

### Example 9

### Affinity Selection of EGFR-Specific Clones

A llama was immunized with a mixture of EGFR ECDs (wild type and vIll mutant), and cell lysates (lung adenocarcinoma and glioblastoma). Lymphocytes were isolated and a llama-VHH domain phagemid library was constructed. The library contained approximately 10⁷ recombinants, and sequencing of 20 random isolates revealed each to be unique and all to have the characteristic framework (FR) and complementarity determining (CDRs) of a llama heavy chain variable domain (Woolven et al. (1999) Immunogenetics 50:98-101). Using this library, four rounds of affinity selection by phage display on purified ECDs of EGFR and EGFRvIII were performed. Between Rounds 1 and 3 it was observed that the number of eluted phage (output) as a fraction of total phage added to the well (input) increased 1000-fold for both selections. However, there was no rise in output/input between Round 3 and Round 4; therefore the selection appeared to be complete after Round 3. One hundred ninety phage from the third round of each selection were individually tested by ELISA on the protein against which they were selected, and simultaneously on BSA-blocked plastic. Approximately 50% of the phage tested could be characterized as “strong positives” (i.e., they gave A₄₀₅ readings >1.0; with >15-fold higher signal on target protein over assay background). Another 25% were plastic or BSA binders and the remainder bound target weakly or not at all. As an additional control for non-specific binding, twenty-four of the best binders were also tested on immobilized cyclophilin A, to which no detectable binding occurred.

### Example 10

### Specificity Testing on Cell Lines

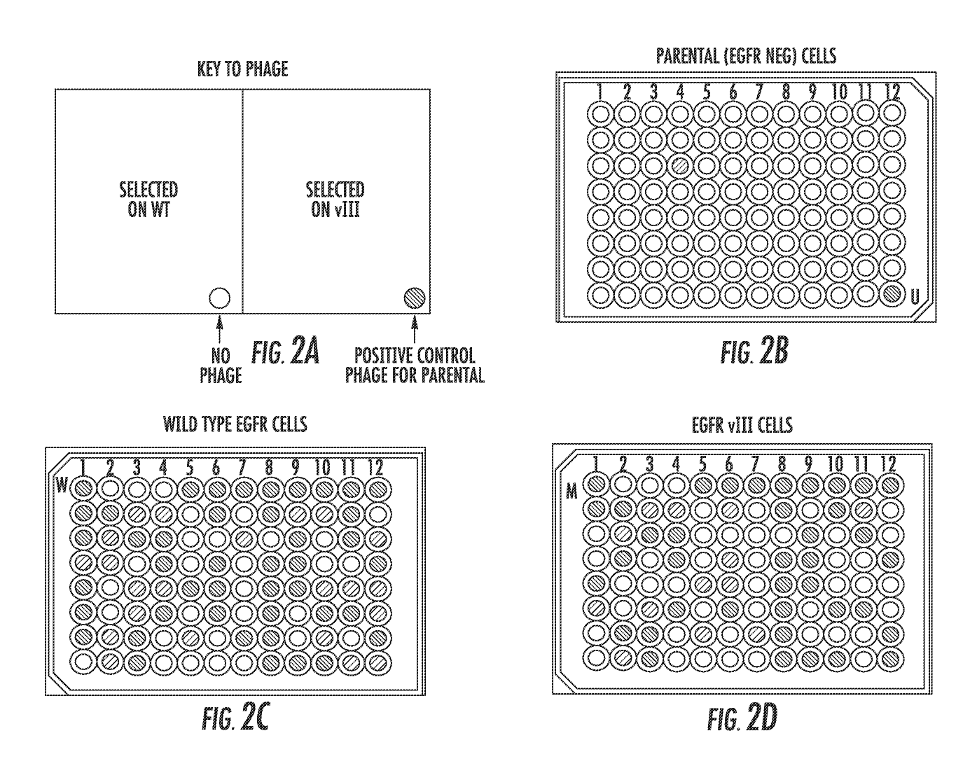

A total of 94 phage that were strongly positive in the EGFR protein ELISA were then tested in a cell-based ELISA. See, FIGS. 2A-2D. In this assay, all the phage were simultaneously tested for binding to mouse NR6 fibroblasts expressing human EGFR(NR6W; FIG. 2C) or human EGFRvIII (NR6M; FIG. 2D), and the parental cell line that expresses no endogenous EGFR(NR6V; FIG. 2B) (Bata et al. (1995) Cell Growth and Differentiation 6:1251-1259). The results can be summarized as follows: (a) 39% of the tested phage could be characterized as “strong positives” (A₄₀₅>1.0 on EGFR-expressing cells; >0.5 on EGFRvIII-expressing cells); (b) most of the phage selected on either type of receptor bind to cells expressing either type of receptor, but only 1 phage binds (weakly) to the parental cells and/or to plastic (FIG. 2B); (c) two phage clones (in wells C1 and G1; FIGS. 2C and 2D) appear to be specific for wild type but not VIII mutant EGFR-expressing cells. Following this experiment, several hundred additional phage were assayed by cell ELISA and EGFR-specific phage were readily identified.

### Example 11

### Competition ELISA Showing Cross-Specificity with EGFR-Specific Mabs

In order to determine whether the VHH-phage have cross-specificity with EGFR-specific mAbs, phage versus mAb competition experiments were performed on fixed glioblastoma cells expressing EGFR. All the anti-EGFR mAbs are specific for the ECD. A wild type EGFR-specific phage (from well C1 in FIG. 2C) was competed by two different anti-EGFR mAbs, F2A2 and H11, but not by anti-EGFR mAbs B10D11 (FIG. 3) or D2C7 (data not shown), an anti-EGFRvIII-specific mAb or an anti-actin mAb. The two competing antibodies, F2A2 and H11, have cross-specificity with each other (data not shown), and the data suggests that this VHH-phage shares the same specificity with them. Conversely, nine other VHH-phage are competed by B10D11 and D2C7 but not by F2A2 and H11.

### Example 12

### Immunoprecipitation of Target with Immobilized VHH Domains

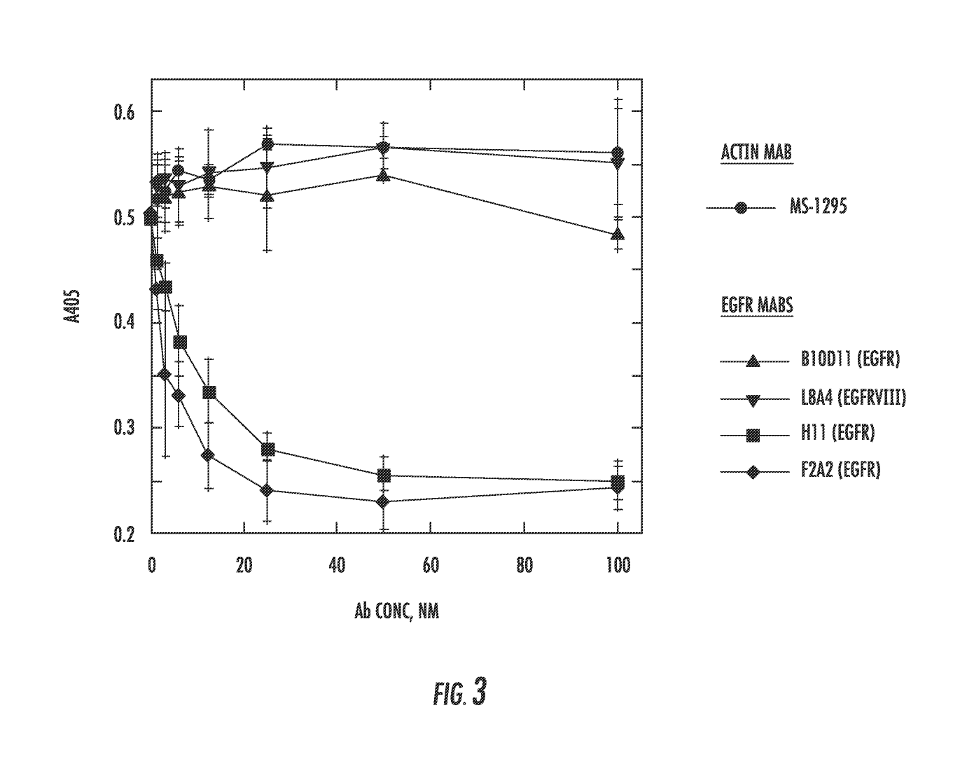

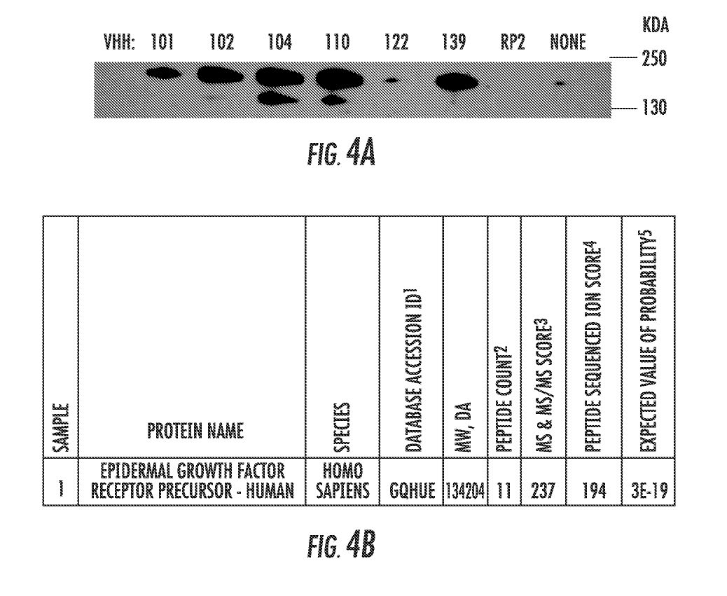

Free VHH domains containing a polyhistidine tag were expressed in and purified from E. coli strain HB2151 by nickel affinity chromatography. VHH domains 101, 102, 104, 110, 122, and 139 and one random VHH clone (RP2; SEQ ID NO: 47) were bound in individual wells of a nickel-chelate microplate; an eighth well received only buffer. NR6W lysate was added to each well and allowed to incubate for 16 hr at 4° C. The lysate was removed, the wells were washed and remaining protein eluted. Samples were subjected to SDS-PAGE and proteins were transferred to a PVDF membrane that was then probed for EGFR. An EGFR-positive band was obtained with almost all of the VHH domains of interest, and no band was seen with the negative controls (FIG. 4A). VHH122, for reasons not yet understood, does not pull down EGFR in the format of this assay. However, as described below, EGFR (visible by Coomassie Blue staining and identity confirmed by peptide sequencing) could be isolated from cell lysates incubated with VHH122 covalently linked to Sepharose beads. In addition, real time association and dissociation of VHH122 with immobilized EGFR ECD could be measured in surface plasmon resonance experiments as discussed below. See, e.g., Examples 13 and 14. Without being bound to any single proposed explanation, it is possible the epitope on EGFR is obscured by the solubilizing detergent in M-PER used in this immunoprecipitation assay. It is also possible that VHH122 immobilized on a flat surface (i.e. in the 96-well microplate) is not able to access the epitope on EGFR, while VHH122 coupled to a sepharose bead is capable of accessing the epitope.

### Example 13

### Identification of protein immunoprecipitated by VHH122

VHH122 and negative control VHH RP2 were each coupled to CNBr-activated Sepharose 4B beads (GE Healthcare) and incubated with NR6W lysate. The beads were washed and protein eluted with SDS-Protein Sample Buffer at 95° C. Eluted protein was subjected to SDS-PAGE and stained with Coomassie Blue; 2 bands, one of an apparent molecular weight of 170,000 and one of an approximate molecular weight of 17,000 were seen in the VHH122 lane. Only the 17,000 molecular weight species (the size of the VHH domain) was seen in the RP2 control lane. The single band unique to the VHH122 lane was excised from the gel. This protein was subjected to tryptic digestion, MALDI-TOF peptide fingerprint analysis and MS/MS sequencing. The analysis identified the immunoprecipitated protein as EGFR (FIG. 4B).

### Example 14

### Specificity of VHH122 for EGFR-expressing cells by flow cytometry

NR6W and NR6V cells (8×10⁵) were mixed with either VHH122 or the random control RP2 at 15 μg/ml and incubated for 1 hr on ice. The cells were washed and stained with fluorescein isothiocyanate (FITC)-conjugated mouse anti-His tag mAb, washed and analyzed in a BD FACScalibur® flow cytometer (BD Biosciences). The results of the flow cytometry experiment are shown in FIG. 5, where the single shifted peak represents VHH122 bound to NR6W cells.

### Example 15

### Sequences, Isoelectric Points and Stability of EGFR-Specific VHH Domains

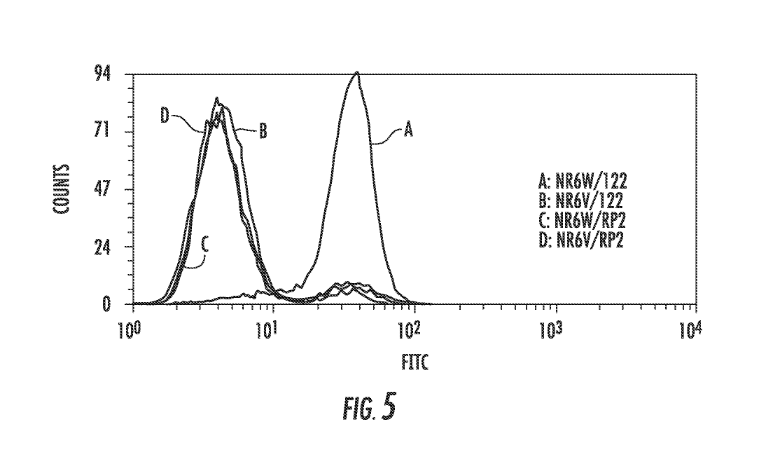

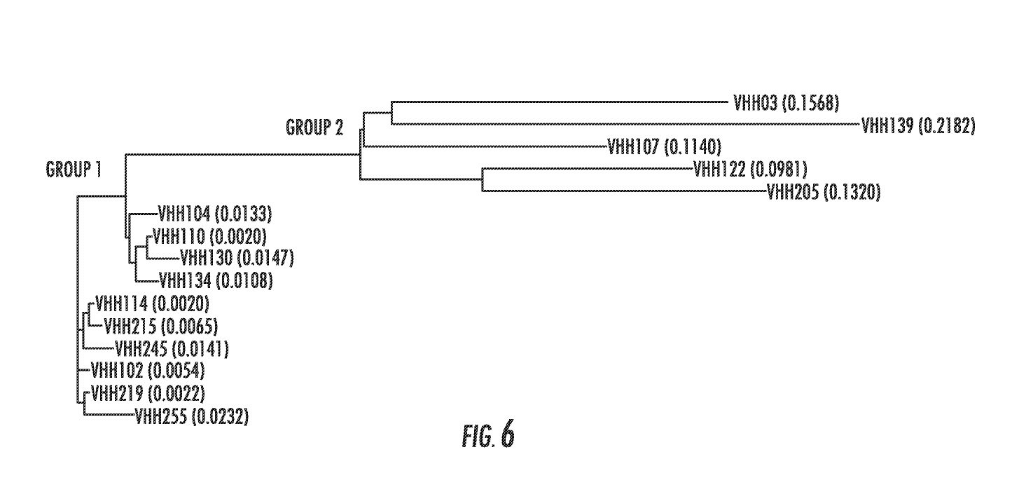

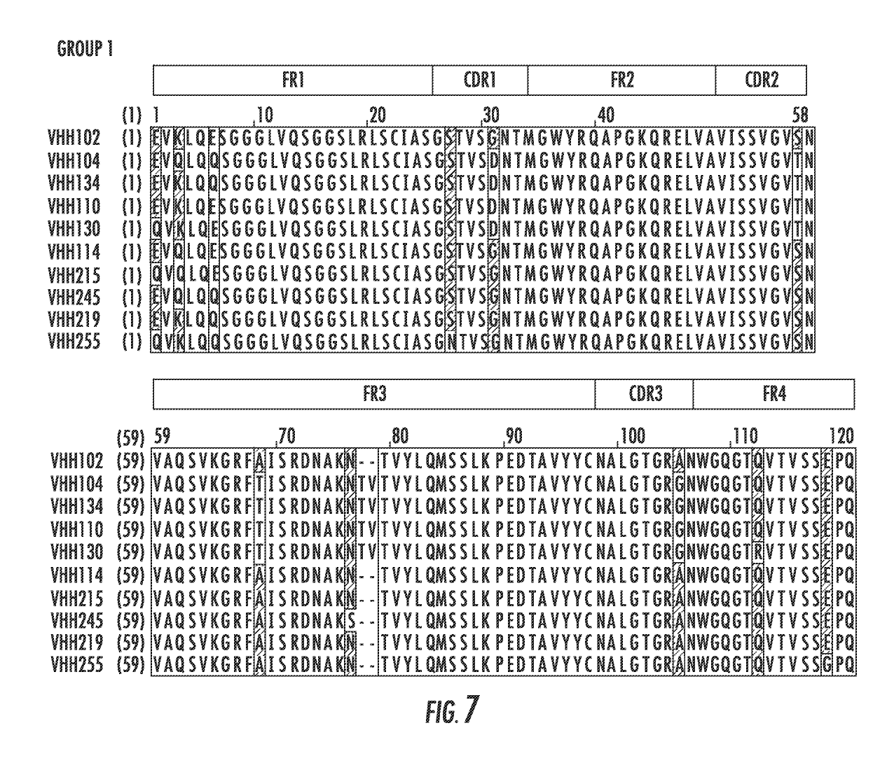

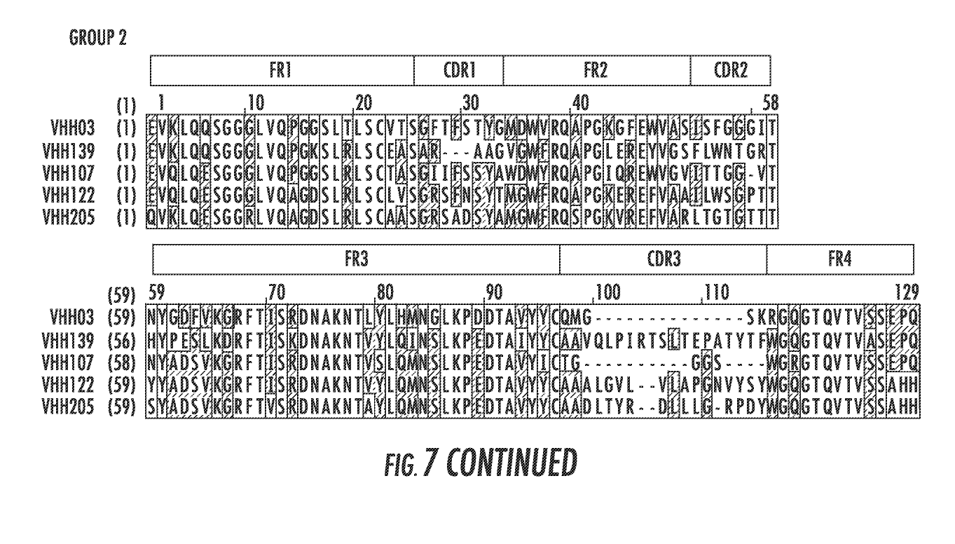

The DNA sequences of VHH domains in the phage with highest apparent affinity by cell ELISA were determined. A total of 167 sequences were obtained, 47 of which were unique. All 47 of these sequences were aligned using the Clustal W function of Vector NTI® (Invitrogen Corp.) and a guide tree of sequence relatedness was constructed using the method of Saitou and Nei (Saitou & Nei (1987) Mol. Biol. Evol. 4:406-425). The guide tree and alignment of a representative subset of 15 sequences are shown in FIGS. 6 and 7, respectively. The EGFR-specific VHH domains fall into 2 groups. Group 1 (33 VHH domains) is highly conserved overall and contains the consensus motifs GSTVS(G/D)NTMG in CDR1, ISSVGVT in CDR2 and NALGTGR(G/A)N in CDR3. (CDR/FR boundaries are demarcated per Harmsen et al. (2000) Mol Immunol 37:579-590). None of these motifs were seen in 20 randomly chosen phage from the 10⁷ member library before selection. Group 2 (13 VHH domains) displays more heterogeneity throughout the molecule, particularly in CDR3, where length of this subdomain varies between 5-19 amino acids. Overall, the sequence identity between any two clones varies between 67% and 99%. The predicted protein molecular weights ranged from 16-18 kDa and calculated isoelectric points ranged from 5.31 to 9.01. The predicted molecular weights, expressed as Daltons, and calculated isoelectric points of some of the VHH domains are listed in Table 3. In order to examine VHH domain stability in serum, purified VHH122 protein was added at a final concentration of 10 μg/ml to either mouse serum (Invitrogen Corp) or normal human serum. As measured by Western blot, VHH122 appeared to be stable to serum proteases (both mouse and human) over the course of 4 hr at 37° C. (FIG. 8). Subsequent experiments demonstrated VHH122 stability to mouse serum proteases over the course of 8 hr at 37° C. In addition, VHH122 retains full EGFR binding activity (as measured in an ELISA with immobilized EGFR ECD) after 1 hour incubation at temperatures ranging from 4° C. to 65° C., but loses all activity upon boiling and cooling to room temperature.

TABLE 3
Predicted Molecular Weight and pI of VHH Domains

| VHH | MW | pI |
|---|---|---|
| 97 | 16,507 | 8.08 |
| 101 | 17,734 | 6.74 |
| 102 | 16,204 | 8.66 |
| 104 | 18,903 | 8.60 |
| 107 | 16,009 | 6.31 |
| 110 | 16,493 | 8.08 |
| 114 | 16,204 | 8.09 |
| 122 | 17,157 | 6.04 |
| 130 | 16,520 | 9.01 |
| 134 | 16,537 | 8.08 |
| 139 | 17,733 | 7.29 |
| 141 | 17,734 | 6.74 |

### Example 16

### Binding Kinetics and Thermodynamics

The ka, kd, and KD values were determined for the binding of two VHH domains to the ECDs of EGFR and EGFRvIII by surface plasmon resonance (SPR). The ECDs were coupled to BIACORE® CM5 chips and VHH domains were sequentially flowed over each protein in the BIACORE® 3000 (GE Healthcare). VHH122 and VHH205 both produced well-defined sensograms from which kinetic and rate constants could be derived; these are shown in Table 4. VHH122 bound to both EGFR and EGFRvIII with roughly equal affinity, approximately 40 nM. Two other VHH domains dissociated rapidly, such that their sensograms could not be fit by the instrument software. The random VHH domain RP2 was also tested and did not bind to either target.

TABLE 4
VHH122 and VHH205 binding to EGFR ECDs by SPR

| | | Conc. | | | | |
|---|---|---|---|---|---|---|
| Receptor | VHH | μM | ka (Ms) | kd (1/s) | KA (1/M) | KD (M) |
| EGFR | 122 | 0.6 | 8.23e4 | 3.18e−3 | 2.58e7 | 3.87e−8 |
| EGFR | 122 | 1.0 | 5.76e4 | 3.33e−3 | 1.73e7 | 5.78e−8 |
| EGFR | 122 | 1.0 | 1.02e5 | 2.07e−3 | 4.95e7 | 2.02e−8 |
| EGFRvIII | 122 | 0.6 | 9.13e4 | 3.04e−3 | 3.01e7 | 3.32e−8 |
| EGFRvIII | 122 | 1.0 | 5.61e4 | 3.08e−3 | 1.82e7 | 5.50e−8 |
| EGFR | 205 | 1.0 | 1.54e5 | 1.15e−2 | 1.34e7 | 7.47e−8 |

### Discussion of Examples 1-16

Monoclonal antibodies are useful but not optimal tumor-targeting agents because high interstitial pressure inside tumors prevents convection of antibodies from blood vessels, and the large size of an IgG (160 kDa) limits its rate of diffusion (Jain, R. K. in Tumour Angiogenesis. (eds. R. Bicknell, C. E. Lewis & N. Ferrara) 45-59 (Oxford University Press, Oxford; 1997)). In addition, mAbs are slowly cleared from the blood with the potential to cause damage to normal tissue and some can be immunogenic. A smaller, non-immunogenic targeting agent with fast tumor infiltration kinetics that retained the high specificity and affinity of an antibody would be of tremendous utility in tumor imaging and therapy. VHH domains of camels and llamas are one-tenth the molecular weight of a conventional antibody yet exhibit equivalent specificity. They are thermodynamically stable (van der Linden et al. (1999) Biochim. Biophys. Acta 1431:37-46), are stable in serum, and appear to be non-immunogenic in humans.

In order to create a resource for the isolation of tumor-specific targeting agents, we immunized a llama with two different types of tumor cell lysates, as well as purified wild type and vIII mutant EGFR ECDs, and constructed a VHH domain phage library. After three rounds of selection on each type of EGFR ECD and assay by ELISA on protein, one-half of all VHH-phage tested were EGFR-specific, validating the approach with respect to this target. Two clones bound only to wild type EGFR and not the mutant, but none of the clones were EGFRvIII specific. Thus it appears that most of the phage might be directed towards epitopes that are common to wild type and vIII mutant EGFR. It is noted that the epitope defined by the vIII mutation is small compared to the size of the protein (Pedersen et al. (2001) Ann Oncol 12:745-760). Thus, additional selection on a peptide spanning the EGFRvIII mutation could yield EGFRvIII-specific VHH domain phage. DNA sequencing and Clustal W analysis of 47 translated coding regions revealed a family of VHH domains that could be split into two major branches due to amino acid sequence similarities in their CDRs. Nine of the phage, some of which were placed in the first branch and some in the second, were competed by the antibodies D2C7 and B10D11 but not by F2A2 and H11, and one phage (wild type EGFR-specific) showed the converse behavior. This result implies that there are 2 binding sites on EGFR that are targeted by phage in the library. The fact that the VHH-phage compete with mAbs for binding to cellular receptor implies that the VHH domains might be recognizing some of the same epitopes as the mAbs. The binding sites for mAbs and VHH domains could also be overlapping or separate but allosteric. The observed sequence variation and wide range of isoelectric points (pl values between 5.3 and 9.3) portends different degrees of physical and/or biological stability between the VHH domains, which might be exploited in development of therapeutic or imaging agents.

The instant disclosure demonstrates that the VHH domains can bind to cells as free antibodies and can be used to pull out the receptor by immunoprecipitation from cell extracts. MALDI-TOF peptide fingerprint analysis and MS/MS sequencing offered direct proof of the identity of the target. It is believed that this is the first direct confirmation of the identity of an integral cell membrane receptor target of a phage displayed antibody. In some embodiments, the methods presented herein can be used to identify the targets of potentially useful VHH domains isolated from the library in the future. Further, in some embodiments the llama phage display library disclosed herein can provide a ready source of probes for EGFR and other tumor cell targets, as demonstrated in the following examples.

### Example 17

### In Vivo Biodistribution and Tumor Imaging Using Radiolabeled VHH Domains

An EGFR-expressing tumor in the mouse was successfully imaged by microPET using ¹²⁴I-labeled VHH122, one of the EGFR-specific, llama-derived antibody fragments described in accordance with the presently disclosed subject matter. VHH122 was labelled with ¹²⁵I and the Kd of [¹²⁵I]VHH122 was determined to be 46 nM (FIG. 9), a value similar to that found for the unlabeled molecule by SPR measurements, indicating that the ¹²⁵I-labeled molecule had not lost binding affinity upon labeling. VHH122 was then labeled with ¹²⁴I (a positron emitter chemically identical to ¹²⁵I) and conducted a pilot study to assess both biodistribution and the feasibility of PET imaging using this probe.

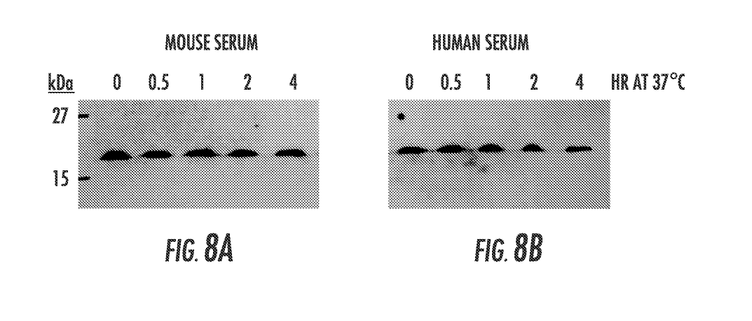

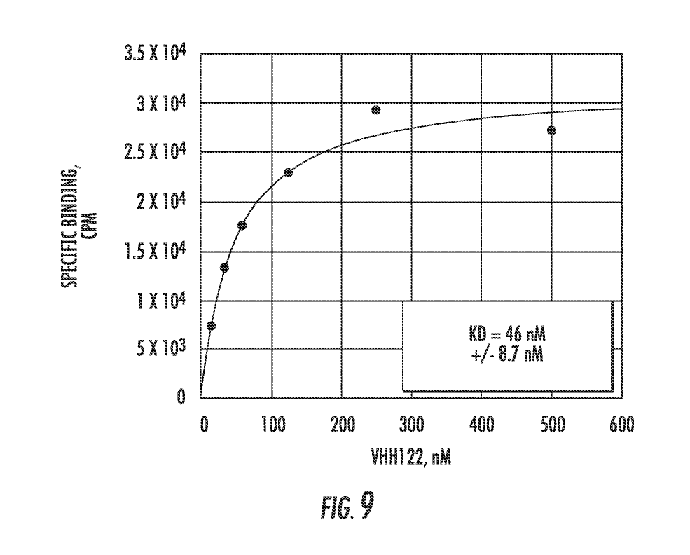

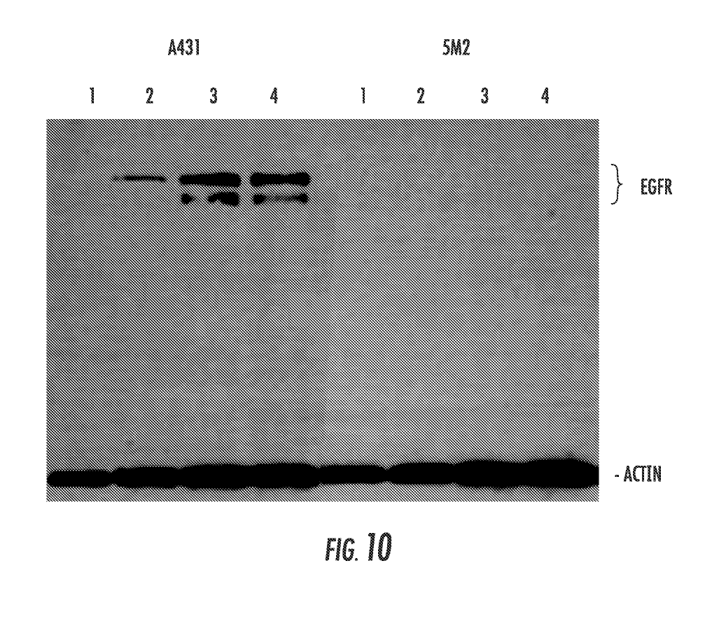

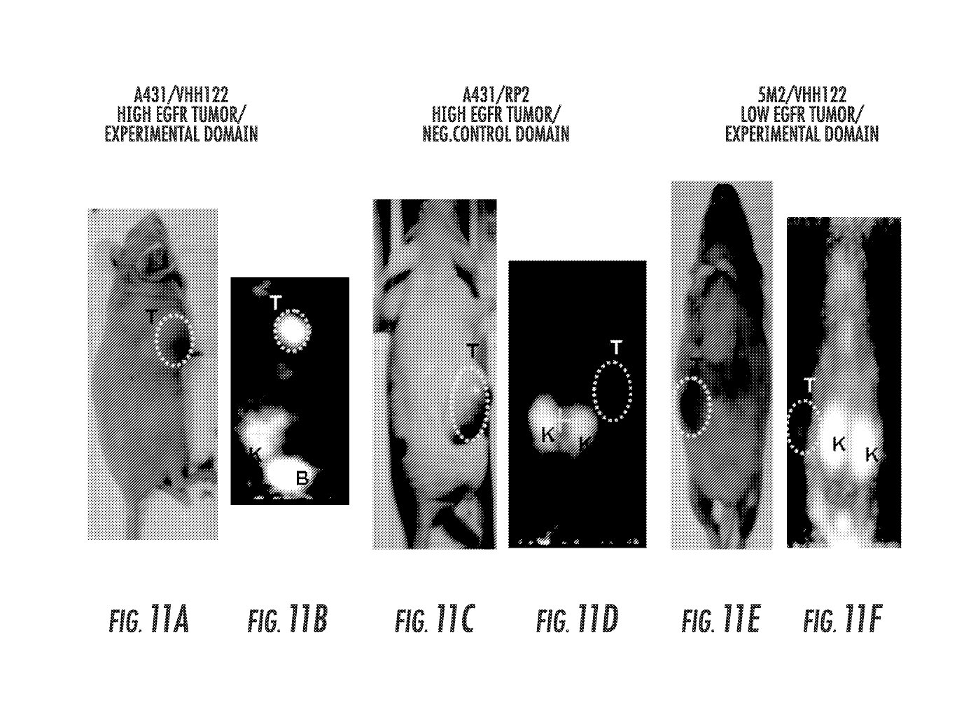

Nude mice were injected subcutaneously with 1×10⁷ A431 (high EGFR) or 5M2 (low EGFR) tumor cells (FIG. 10), and tumors subsequently formed at the sites of injection. Both VHH122 (SEQ ID NO: 17) and RP2 (SEQ ID NO: 47), a control VHH domain that does not bind EGFR, were labeled with ¹²⁴I using iodination beads (Pierce) and purified using desalting spin columns. Three mice were sequentially anesthetized and then each was injected via tail vein with labeled protein. At predetermined times the mice were placed on the microPET scanner. One mouse bearing an A431 tumor and one bearing a 5M2 tumor were each injected with the experimental [¹²⁴I]VHH122; one additional mouse bearing an A431 tumor was injected with the control VHH domain [¹²⁴I]RP2. Images were acquired for 15 minutes at 45 minutes after injection (FIG. 11). The animals were then sacrificed and dissected and radioactivity in organs was quantified in a gamma counter (Table 5).

TABLE 5
Tumor:organ ratios of ¹²⁴I accumulation in the
3 animals used in the imaging experiments

| | Tumor:Organ Ratios | | |
|---|---|---|---|
| Organ | A431-VHH | A431-Con | 5M2-VHH |
| Liver | 2.4 | 0.4 | 0.4 |
| Spleen | 16 | 3.1 | 4.2 |
| Kidneys | 2.1 | 0.6 | 0.1 |
| Thyroid | 1.0 | 0.5 | 3.7 |
| Muscle | 47 | 6.8 | 15 |
| Blood | 8.9 | 1.9 | n.d. |

[¹²⁴I]VHH122 clearly imaged the A431 tumor but not the 5M2 tumor, consistent with the relative levels of EGFR in the two cell lines. Unlike [¹²⁴I]VHH122, [¹²⁴I]RP2 did not image the A431 tumor, implicating EGFR-specificity in tumor accumulation of [¹²⁴I]VHH122. [¹²⁴I]VHH122 displayed an A431 tumor:muscle ratio of 47:1, versus 15:1 for the 5M2 tumor, again consistent with EGFR-specificity. [¹²⁴I]RP2 displayed an A431 tumor:muscle ratio of 7:1, probably due to leakiness of tumor blood vessels; however, this degree of accumulation did not result in high contrast tumor images. Both VHH domains appeared to be rapidly excreted through by the kidneys.

These results provide a suitable foundation and guidance for one of ordinary skill in the art to perform additional in vivo methods, based on the presently disclosed subject matter, in animal models and/or human subjects.

### Example 18

### Tumor Imaging In Vivo in Human Subjects Using EGFR-Specific Radiolabeled VHH domains

Based on animal biodistribution, imaging and dosimetry data obtained in accordance with the presently disclosed subject matter, candidate VHH domains are evaluated to determine the best VHH domain for clinical studies. In some embodiments, the pharmicokinetics (PK), biodistribution, human dosimetry, and/or safety of a single injection of labelled VHH domain, e.g. ¹⁸F-labeled VHH domain or ¹²⁴I-labeled VHH domain, in a limited number of patients with lung cancer is determined. In some embodiments, labeled VHH domain uptake in a primary lesion with EGFR status is evaluated, as determined by immunohistochemistry (IHC) of the primary tumor. This can allow for the correlation of labeled VHH domain uptake as measured by PET and EGFR status in lung cancer.

By way of example and not limitation, a Phase 1 Clinical Trial is performed as follows. A number of patients, e.g. 15 patients, or as many as would be necessary based on the knowledge and experience of one of ordinary skill in the art, with a new diagnosis of advanced stage III-IV non-small cell lung cancer (NSCLC) are recruited

A suitable VHH domain, e.g. VHH122, radiolabeled with ¹⁸F or ¹²⁴I, or any suitable radiolabel as would be appreciated by one of ordinary skill in the art upon a review of the instant disclosure, as described above is used as an imaging probe. Patients are administered a single dose intravenously, with the activity level based on dosimetry data obtained in the above animal experiments, or similarly conducted experiments. The Chemistry, Manufacturing, and Controls (CRC) for preparation of an imaging probe follows all FDA guidelines meeting their current good manufacturing practice (CGMP) regulations. This can include sterility and pyrogenicity testing.

PET-CT imaging with the labeled VHH domain is performed on a suitable PET-CT scanner from the head through the pelvis to determine biodistribution throughout the body. The CT scan is performed for anatomic localization and attenuation-correction of the PET images. Following intravenous injection of approximately 5-10 mCi, or any other suitable dose of probe extrapolated from the presently disclosed subject matter or in vivo studies conducted in accordance with the presently disclosed subject matter, serial PET emission images are acquired at, for example, 1, 30, 60 and 120 minutes after injection to determine PK and biodistribution data. The serial imaging times are modified as necessary in accordance with the presently disclosed subject matter and as would be appreciated by one of ordinary skill in the art upon a review of the instant disclosure. VHH protein dose on tumor uptake and image contrast are investigated and adjusted as necessary and as would be appreciated by one of ordinary skill in the art in order to optimize image quality and tumor uptake, upon a review of the instant disclosure.

Blood samples for determining radioactivity and analysis of labeled catabolites are performed in a select number of patients, e.g. five patients, with samples obtained at the time of administration and at 1, 3, 5, 10, 30, 45, 60, 90 and 120 minutes post-injection. Serum is analyzed by HPLC and SDS-PAGE for radioactive metabolites. VHH protein dose is investigated and adjusted as necessary and as would be appreciated by one of ordinary skill in the art upon a review of the instant disclosure in order to minimize imaging of normal EGFR-expressing tissue, e.g. liver and other organs, while maximizing imaging of EGFR-expressing tumors.

All PET-CT images are evaluated without knowledge of EGFR status. All regions of labeled VHH domain uptake are compared to the corresponding anatomic CT images and recorded. Standardized uptake values (SUVₘₑₐₙ and SUVₘₐₓ) are determined for the primary lesion and all major organs including lungs, heart, liver, spleen, bone marrow, kidneys, bladder, brain, and bowel. These data are used to establish the PK, biodistribution and dosimetry of the radio-labeled VHH domain.

Uptake in the primary lesion is determined by each of three methods at the designated time points: (1) area under the curve of absolute uptake, (2) SUVₘₑₐₙ, and (3) SUVₘₐₓ. These uptake data are correlated with the EGFR status as determined by IHC of the primary tumor at the time of diagnosis.

EGFR IHC is performed using a monoclonal EGFR antibody (e.g., Clone 31G7, Zymed Laboratories Inc., South San Francisco, Calif., United States of America) according to the manufacturer's instructions. A pulmonary pathologist is blinded to clinical information and disease status, and can separately score cytoplasmic and nuclear staining. The traditional scoring scale from 0 (no staining) to 3+ (strong staining) is used. The percentage of total tumor cells with any amount of staining is also recorded to provide an indication of the heterogeneity of EGFR expression within the tumor. An H-score is calculated by multiplying the staining score by the percentage of cells staining.

The primary clinical objectives for a study of this nature can optionally be to determine PK, biodistribution, and/or safety of the radio-labeled VHH domain. Given that the occurrence of any adverse event attributed to the treatment is deemed to be unacceptable in this embodiment, there are no statistical considerations for the safety aim.

For each of the patients, SUV is collected at 4 time points (1, 30, 60, and 120 minutes post-injection). The EGFR status of each patient's tumor is determined from the primary tumor by IHC. The association between IHC H-score and the highest observed SUV for the primary tumor is investigated using the Spearman rank-correlation (Hajek et al. (1999) Theory of Rank Tests, 2nd edition.: Academic Press). For illustration of the power, a Gaussian copula (Nelsen, R. (1999) Introduction to Copulas. Springer-Verlag) with standard normal marginals (i.e., a standard bivariate normal distribution) is used to generate the joint distribution. The dependence parameter for the copula can be chosen so as to correspond to a Spearman correlation of rho=0.6, 0.65 and 0.7. The power at the nominal two-sided level of 0.05 is 0.63 (rho=0.6), 0.73 (rho=0.65) and 0.81 (rho=0.7). The relationship can also be presented graphically using a scatter plot.

Sera is collected at 9 time points for a select number of patients, e.g., 5 of the 15 patients, in order to study the PK of the VHH antibody. Individual longitudinal serum concentration profiles of the antibody and its metabolites are illustrated graphically. The mean along with a confidence interval, assuming that the concentrations are normally distributed, is annotated on this plot.

### Example 19

### Re-Screening the Library for VHH Domains of Higher Affinity

In some embodiments of the presently disclosed subject matter it might be desirable to increase the tumor accumulation of small antibodies such as VHH domains by improving affinity. In some embodiments it is estimated that if accumulation peaks within a time frame of 1-2 hours post-injection this should be sufficient for imaging. Therefore, if one of ordinary skill in the art in performing the presently disclosed subject matter discovers that the first particular VHH domain does not accumulate at sufficient levels for optimal imaging, optionally higher affinity molecules are evaluated. Such optimization of VHH domains with sufficient affinity is believed to be in accordance with the presently disclosed subject matter and within the skill of one of ordinary skill in the art, particularly in view of the instant disclosure.

In some embodiments the source of such additional VHH domains from which to choose can be the llama VHH domain library disclosed herein. The robust immune response observed in the llama after inoculation of EGFR ECD antigen suggests a wide variety of antibodies of differing affinities represented in the library. One of ordinary skill is well equipped, based on the instant disclosure and knowledge in the art, to rescreen the library by performing phage affinity selections to identify suitable VHH domains with a desired affinity and imaging capability.

In order to isolate phage of higher affinity, the phage display protocol described herein can optionally be altered in at least two ways. First, the time of phage incubation with the target protein is shortened from 3 hr to 30 min to select for phage with faster on rates. Second, 3 hr-long wash steps is introduced after the binding step in order to select for phage with slow off rates. In some embodiments, 10 pmole of EGFR ECD in 0.1 M NaHCO₃ pH 8.5 is bound in a microplate well at 4° C. overnight. The well is blocked for 1 hr with 1% BSA in the same buffer. The well is washed with TBST and ˜10¹¹ library phage added. The phage are allowed to bind the target for 30 min at room temperature, after which they are removed. The plate is washed 8 times with 250 μl PBST quickly to remove bulk phage, and then incubated 3 times successively with 250 μl PBST for 1 hr each at room temperature. Phage remaining on the target are eluted by alternating acid and base treatment. E. coli TG1 is infected with the eluted phage for amplification and rescue. In some embodiments three rounds of selection are completed followed by assessment of the specificity and relative affinities of individual phage.

Phage are assessed for specificity by cell ELISA on NR6W (EGFR-positive) vs. NR6V (EGFR-negative) cells as described herein. NR6W-specific phage are then assessed for relative affinity by ELISA on NR6W cells. Phage are titered, diluted and placed in wells with glutaraldehyde-fixed cells. Phage binding is detected with anti-M13-HRP and absorbance versus phage number is plotted. Phage 122, the parent phage of VHH122, is the standard against which other phage are compared. Higher affinity phage give a higher absorbance value per given quantity of phage, in the linear portion of the graph. Free VHH proteins corresponding to high affinity phage are expressed and characterized as described herein. Kinetic rate constants and the dissociation constant of newly isolated VHH domains are calculated as disclosed herein.

### Example 20

### Increasing Molecular Weight to Improve Affinity and/or Half-Life

In some embodiments tumor accumulation of a small antibody is improved by increasing its molecular weight by dimerization. This can have the dual effect of increasing the affinity through avidity and also slowing the loss of agent through kidney uptake, although the dimeric constructs provided herein are well below the glomerular filtration limit of ˜65,000 (Brenner et al. (1976) Physiol Rev 56:502-534). In addition to making homodimeric constructs, heterodimeric constructs comprising two different VHH domains are constructed. In order to confer conformational flexibility on the molecule, the two domains are connected by a linker, such as but not limited to, a 29 amino acid linker derived from the natural hinge region of one subclass of llama IgG.

VHH dimeric constructs can comprise two copies of a single VHH domain separated by the upper hinge of llama IgG2 (Vu et al. (1997) Mol Immunol, 34:1121-1131). First, a linker encoding this hinge flanked by SfiI and NotI cohesive ends is cloned into the SfiI and NotI sites of pHEN1-H6. The SfiI and NotI sites that flank the linker then serve as cloning sites for two copies of the VHH domain of interest. The phagemid clone is amplified with a high fidelity polymerase using each of two sets of primers such that the final product can contain 2 flanking SfiI or 2 flanking NotI sites. First one, then the other PCR product is cloned and the sequence of the entire insert confirmed using 2 flanking and two internal (inter-linker) primers. The final construct can comprise, consist essentially of, or even consist of the following elements in frame in the pHEN1 backbone (FIG. 1): P_{lac}—PelB leader—SfiI site—VHH coding sequence 1—Hinge—VHH coding sequence 2—Not I site—His₆—c-myc. Once the hinge region is cloned, any two VHH domains are cloned into the vector on either side. However, since each VHH cloning step is not directional, the orientation of clones is determined by restriction enzyme analysis. Since the SfiI site is within the PeIB leader, only one orientation of VHH coding sequence I recreates this element. Once the constructs are made, the recombinant proteins are expressed, purified and characterized as described herein above.

### REFERENCES

Aboud-Pirak et al. (1989) Biochem. Pharmacol. 38:641-648.
Alexay et al. (1996) The PCT International Society of Optical Engineering 2705/63.
Barbas et al. “Phage Display: A Laboratory Manual,” (Cold Spring Harbor Laboratory Press, Cold Spring Harbor, N.Y.; 2001).
Batra et al. (1995) Cell Growth and Differentiation 6:1251-1259.
Bauminger & Wilchek (1980) Meth. Enzymol. 70:151-159.
Bepler et al. (1988) Differentiation 37, 158-171.
Boerman et al. (2000) Semin Nucl Med 30:195-208.
Brenner et al. (1976) Physiol. Rev. 56: 502-534.
Buchsbaum et al. (1995) Cancer Res 55:5881s-5887s.
Chattopadhyay et al. (2001) Nucl. Med. Biol. 28:741-744.
Cheng (1996) Hum. Gene Ther. 7:275-282.
Chowdhury et al. (1999) Nat. Biotechnol. 17:568-572.
Coatney (2001) Ilar J 42:233-247.
Cortez-Retamozo et al. (2002) Int J. Cancer 98, 456-462.
De Genst et al. (2006) Proc. Natl. Acad. Sci. U.S.A. 103:4586-4591.
Dekker et al. (2003) J. Virol. 77:12132-12139.
Desmyter et al. (2001) J. Biol. Chem. 276:26285-26290.
Dewanjee et al. (1994) J. Nucl. Med. 35:1054-1063.
Dikic (2003) Biochem Soc. Trans. 31:1178-1181.
Dillman et al. (1989) Mol. Biother. 1:250-255.
Dumoulin et al. (2002) Protein Sci. 11:500-515.
European Patent No. 0 439 095
Falcioni et al. (1989) Mol. Carcinog. 2:361-368.
Fischer et al. (2003) Biochem. Soc. Trans. 31:1203-1208.
Fjalling et al. (1996) J. Nucl. Med. 37:1519-1521).
Fraser (1996) Meth Cell Biol 51:147-160.
Garcia de Palazzo et al. (1993) Cancer Research 53:3217-3220.
Goldman et al. (1997) Cancer Res 57:1447-1451.
Hamers-Casterman et al. (1993) Nature 363:446-448.
Harmsen et al. (2000) Mol. Immunol. 37:579-590.
Hartmann et al. (1994) Cancer Res 54:4362-4370.
Henn et al. (1993) J Med Chem 36:1570-1579.
Heredia et al. (1991) J Neurosci Meth 36:17-25.
Hirsch et al. (2003) Lung Cancer 41, Suppl 1:S29-42.
Hnatowich et al. (1996) J. Pharmacol. Exp. Ther. 276:326-334.
Hogrefe et al. (2002) Biotechniques 33:1158-1165.
Hoogenboom et al. Nucleic Acids Res. 19:4133-4137 (1991).
Hwang et al. (2005) Methods 36:3-10.
Hajek et al., “Theory of Rank Tests,” 2nd edition, Academic Press, 1999.
Jain, R. K., “Vascular and interstitial physiology of tumours: role in cancer detection and treatment,” In: R. Bicknell, C. E. Lewis, and N. Ferrara (eds.), Tumour Angiogenesis, pp. 45-59, Oxford: Oxford University Press, 1997.
Jain, R. K. in Tumour Angiogenesis. (eds. R. Bicknell, C. E. Lewis & N. Ferrara) 45-59 (Oxford University Press, Oxford; 1997).
Kim et al. (1999) Exp. Cell Res. 253:78-87.
Krauer et al. (1992) Cancer Res 52:132-137.
Krenning & de Jong (2000) Ann Oncol 11:267-271.
Kuan et al. (2000) Brain Tumor Pathol. 17:71-78.
Kwekkeboom et al. (2000) J Nucl Med 41:1704-1713.
Lau et al. (1995) Bioorg. Med. Chem. 3:1299-1304.
Lees (2001) Semin Ultrasound CT MR 22:85-105.
Licha et al. (2000) Photochem Photobiol 72:392-398.
Lorenzo et al. (2003) Clin. Prostate Cancer 2:50-57.
Lynch et al. (2004) N. Engl. J. Med. 350:2129-2139.
Maihie et al. (2002) Cancer Treat. Res. 107:247-258.
Manome et al. (1994) Cancer Res 54:5408-5413.
Meert et al. (2003) Eur. Respir. J. 21:611-615.
Mountain, C. F. Revisions in the International System for Staging Lung Cancer. Chest, 111: 1710-1717, 1997.
Muruganandam et al. (2002) Faseb J. 16:240-242.
Nelsen, R., “Introduction to Copulas,” Springer-Verlag, 1999.
Neri et al. (1997) Nat. Biotechnol. 15:1271-1275.
Nabel (1997) Vectors for Gene Therapy. In Current Protocols in Human Genetics, John Wiley & Sons, New York.
O'Donovan et al. (2007) Anticancer Res. 27:1285-1294.
Okamoto et al. (2003) Cancer Sci. 94:50-56.
Padron et al. (2007) Cancer Res. 67:7695-7702.
Park et al. (1997) Adv. Pharmacol. 40:399-435.
Pasqualini et al. (1997) Nat. Biotechnol. 15:542-546.
PCT International Publication No. WO 98/10795.
Pedersen et al. (2001) Ann. Oncol. 12:745-760.
Piyathilake et al. (2002) Clin. Cancer Res. 8:734-744.
Pomper & Port (2000) Magn Reson Imaging Clin N Am 8:691-713.
Ponten et al. (1968) Acta Pathol. Microbiol. Scand. 74:465-486.
Ragnarson et al. (1992) Histochemistry 97:329-333.
Roovers et al. (2007) Cancer Immunol. Immunother. 56:303-317.
Rowland et al. (1993) Cancer Immunol. Immunother. 37:195-202).
Rovaris et al. (2001) J Neurol Sci 186 Suppl 1:S3-9.
Saerens et al. (2005) J. Mol. Biol. 352:597-607.
Sagiuchi et al. (2001) Ann. Nucl. Med. 15:267-270.
Saitou et al. (1987) Mol. Biol. Evol. 4:406-425.
Saltzman & Fung (1997) Adv Drug Deliv Rev 26:209-230).
Shawler et al. (1988) J. Biol. Response Mod. 7:608-618).
Schechter et al. (1991) Intl. J. Cancer 48:167-172).
Schlessinger, J. (2002) Cell 110:669-672.
Selvaggi et al. (2004) Ann. Oncol. 15:28-32.
Shen et al. (1993) Magn Reson Med 29:599-604.
Sheriff et al. (1996) Nat. Struct. Biol. 3:733-736.
Shih et al. (1994) Cancer Immunol. Immunother. 38:92-98.
Schwendener (1992) Chimia 46:69-77.
Sivam et al. (1995) Cancer Res. 55:2352-2356.
Smyth et al. (1987) Immunol Cell Biol 65:315-321).
Starling et al. (1992) Bioconjug Chem 3:315-322).
Teicher, B. A. (ed.) “Physiological resistance to the treatment of solid tumors,” New York, Marcel Dekker, Inc. (1993).
Tavitian et al. (1998) Nat. Med. 4:467-471.
Ullrich et al. (1984) Nature 309:418-425.
U.S. Pat. No. 4,376,110.
U.S. Pat. No. 4,551,482.
U.S. Pat. No. 5,088,499.
U.S. Pat. No. 5,147,631.
U.S. Pat. No. 5,490,840
U.S. Pat. No. 5,510,103
U.S. Pat. No. 5,651,991.
U.S. Pat. No. 5,688,931.
U.S. Pat. No. 5,707,605.
U.S. Pat. No. 5,714,166.
U.S. Pat. No. 5,738,837.
U.S. Pat. No. 5,786,387.
U.S. Pat. No. 5,855,900
U.S. Pat. No. 5,858,410.
U.S. Pat. No. 5,865,754.
U.S. Pat. No. 5,922,356.
U.S. Pat. No. 5,922,545.
U.S. Pat. No. 5,994,392.
U.S. Pat. No. 6,024,938.
U.S. Pat. No. 6,071,890.
U.S. Pat. No. 6,080,384.
U.S. Pat. No. 6,083,486.
U.S. Pat. No. 6,106,866.
U.S. Pat. No. 6,221,018.
U.S. Pat. No. 6,231,834.
U.S. Pat. No. 6,245,318.
U.S. Pat. No. 6,246,901.
U.S. Pat. No. 6,254,852.
van der Linden et al. (2000) J. Immunol. Methods 240:185-195.
van der Linden et al. (1999) Biochim. Biophys. Acta 1431:37-46.
Vinogradov et al. (1996) Biophys J 70:1609-1617.
Virgolini et al. (2001) Q J Nucl Med 45:153-159.
Vu et al. (1997) Mol Immunol, 34:1121-1131.
Weissleder et al. (1992) Magn Reson Q 8:55-63.
Weissleder et al. (1999) Nat Biotechnol 17:375-378.
Wikstrand et al. (1995) Cancer Research 55:3140-3148.
Wilbur (1992) Bioconjug Chem 3:433-470.
Woolyen et al. (1999) Immunogenetics 50:98-101.
Yau et al. (2005) J. Immunol. Methods 297:213-224.
Yoo et al. (1997) J Nucl Med 38:294-300).
Yuhas et al. (1974) Cancer Res. 34:2005-2010.
Yuhas et al. (1974) Cancer Res. 34:722-728.

All references listed in the instant disclosure, including but not limited to all patents, patent applications, and scientific journals are incorporated herein by reference in their entireties to the extent that they supplement, explain, provide a background for or teach methodology, techniques and/or compositions employed herein.

It will be understood that various details of the presently disclosed subject matter can be changed without departing from the scope of the present disclosure. Furthermore, the foregoing description is for the purpose of illustration only, and not for the purpose of limitation

What is claimed is:

1. A composition for targeting of cancer cells, wherein the composition comprises one or more targeting ligands comprising an antibody fragment, wherein the antibody fragment comprises a VHH domain comprising a sequence as set forth in SEQ ID NOs.: 1-46, or a variant or a derivative thereof.

2. The composition of claim 1, wherein the antibody fragment, or variant thereof, is humanized.

3. The composition of claim 1, wherein the one or more targeting ligands bind to one or more tumor types selected from among bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head, a neck tumor, and a solid tumor.

4. The composition of claim 1, wherein the composition further comprises a detectable label, a therapeutic agent, a carrier, or combinations thereof.

5. The composition of claim 4, wherein the detectable label is an in vivo detectable label, which optionally can be detected using magnetic resonance imaging, scintigraphic imaging, ultrasound, or fluorescence.

6. The composition of claim 5, wherein the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

7. The composition of claim 4, wherein the therapeutic agent is selected from the group consisting of a radionuclide, a cytotoxin, and a chemotherapeutic agent.

8. The composition of claim 4, wherein the carrier is selected from the group consisting of a liposome, a microcapsule, and combinations thereof.

9. The composition of claim 1, wherein the targeting ligand itself acts as a therapeutic agent.

10. A method for delivery of a composition to a target tissue in a subject, the method comprising: administering to the subject a therapeutic composition, a diagnostic composition, or a combination thereof, wherein the therapeutic composition, diagnostic composition, or combination thereof, comprises one or more targeting ligands comprising an antibody fragment, wherein the antibody fragment comprises a VHH domain comprising a sequence as set forth in SEQ ID NOs.: 1-46, or a variant or a derivative thereof, whereby the composition is selectively targeted to the target tissue.

11. The method of claim 10, wherein the antibody fragment, or variant thereof, is humanized.

12. The method of claim 10, wherein the composition further comprises a detectable label, a therapeutic agent, a carrier, or combinations thereof.

13. The method of claim 12, wherein the detectable label is an in vivo detectable label, which optionally can be detected using magnetic resonance imaging, scintigraphic imaging, ultrasound, or fluorescence.

14. The method of claim 13, wherein the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

15. The method of claim 12, wherein the therapeutic agent is selected from the group consisting of a radionuclide, a cytotoxin, and a chemotherapeutic agent.

16. The method of claim 10, wherein the targeting ligand itself acts as a therapeutic agent.

17. The method of claim 12, wherein the carrier is selected from the group consisting of a liposome, a microcapsule, and combinations thereof.

18. The method of claim 10, wherein the target tissue comprises a tumor.

19. The method of claim 18, wherein the tumor is a primary or a metastasized tumor.

20. The method of claim 18, wherein the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor.

21. The method of claim 10, wherein the subject is a warm-blooded vertebrate.

22. A method for imaging a target tissue in a subject, the method comprising: administering to the subject a composition comprising one or more targeting ligands comprising an antibody fragment, wherein the antibody fragment comprises a VHH domain comprising a sequence as set forth in SEQ ID NOs.: 1-46, or a variant or a derivative thereof, wherein the composition further comprises an in vivo detectable label; and detecting the composition.

23. The method of claim 22, wherein the antibody fragment, or variant thereof, is humanized.

24. The method of claim 22, wherein the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³mindium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

25. The method of claim 22, wherein detecting the composition comprises detecting the in vivo detectable label using magnetic resonance imaging, scintigraphic imaging, ultrasound, or fluorescence.

26. The method of claim 22, wherein the composition further comprises a therapeutic agent, a carrier, or combinations thereof.

27. The method of claim 26, wherein the therapeutic agent is selected from the group consisting of a radionuclide, a cytotoxin, and a chemotherapeutic agent.

28. The method of claim 22, wherein the targeting ligand itself acts as a therapeutic agent.

29. The method of claim 26, wherein the carrier is selected from the group consisting of a liposome, a microcapsule, and combinations thereof.

30. The method of claim 22, wherein the target tissue comprises a tumor.

31. The method of claim 30, wherein the tumor is a primary or a metastasized tumor.

32. The method of claim 30, wherein the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor.

33. The method of claim 22, wherein the subject is a warm-blooded vertebrate.

34. A method for treating a tumor in a subject, the method comprising: providing a subject with a tumor; and administering to the subject a therapeutic composition comprising one or more targeting ligands comprising an antibody fragment, wherein the antibody fragment comprises a VHH domain comprising a sequence as set forth in SEQ ID NOs.: 1-46, or a variant or a derivative thereof.

35. The method of claim 34, wherein the antibody fragment, or variant thereof, is humanized.

36. The method of claim 34, wherein the composition further comprises a therapeutic agent, detectable label, a carrier, or combinations thereof.

37. The method of claim 36, wherein the detectable label is an in vivo detectable label, which optionally can be detected using magnetic resonance imaging, scintigraphic imaging, ultrasound, or fluorescence.

38. The method of claim 36, wherein the in vivo detectable label comprises a radionuclide label selected from the group consisting of: ¹⁸fluorine, ⁶⁴copper, ⁶⁵copper, ⁶⁷gallium, ⁶⁸gallium, ⁷⁷bromine, ^{80m}bromine, ⁹⁵ruthenium, ⁹⁷ruthenium, ¹⁰³ruthenium, ¹⁰⁵ruthenium, ^{99m}technetium, ¹⁰⁷mercury, ²⁰³mercury, ¹²³iodine, ¹²⁴iodine, ¹²⁵iodine, ¹²⁶iodine, ¹³¹iodine, ¹³³iodine, ¹¹¹indium, ¹¹³indium, ^{99m}rhenium, ¹⁰⁵rhenium, ¹⁰¹rhenium, ¹⁸⁶rhenium, ¹⁸⁸rhenium, ¹²¹mtellurium, ^{122m}tellurium, ^{125m}tellurium, ¹⁶⁵thulium, ¹⁶⁷thulium, and ¹⁶⁸thulium.

39. The method of claim 36, wherein the carrier is selected from the group consisting of a liposome, a microcapsule, and combinations thereof.

40. The method of claim 36, wherein the therapeutic agent is selected from the group consisting of a radionuclide, a cytotoxin, and a chemotherapeutic agent.

41. The method of claim 34, wherein the targeting ligand itself acts as a therapeutic agent.

42. The method of claim 34, wherein the tumor is a primary or a metastasized tumor.

43. The method of claim 34, wherein the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor.

44. The method of claim 34, wherein the tumor over-expresses epidermal growth factor receptor (EGFR).

45. The method of claim 34, wherein the subject is a warm-blooded vertebrate.

46. A method for determining the presence of a tumor, the method comprising: biopsying a suspected tumor; contacting the biopsy of the suspected tumor with a composition comprising an antibody fragment, wherein the antibody fragment comprises a VHH domain comprising a sequence as set forth in SEQ ID NOs.: 1-46, or a variant or a derivative thereof, wherein the composition further comprises a detectable label; and detecting the composition bound to the biopsy of the suspected tumor, whereby the detection of the composition on the biopsy of the suspected tumor determines that the suspected tumor is a tumor.

47. The method of claim 46, wherein the antibody fragment, or variant thereof, is humanized.

48. The method of claim 46, wherein the composition further comprises a carrier selected from the group consisting of a liposome, a microcapsule, and combinations thereof.

49. The method of claim 46, wherein the detectable label is a fluorescent or radioactive label.

50. The method of claim 46, wherein the detection of the composition comprises detecting the detectable label using autoradiography or fluorescence.

51. The method of claim 46, further comprising rinsing the biopsy of the suspected tumor to remove unbound targeting ligands from the biopsy of the suspected tumor.

52. The method of claim 46, wherein the tumor is a primary or a metastasized tumor.

53. The method of claim 46, wherein the tumor is selected from the group consisting of: bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor.

54. The method of claim 46, wherein the tumor over-expresses epidermal growth factor receptor (EGFR).

55. The method of claim 46, wherein the biopsy is obtained from a warm-blooded vertebrate.
